(19) United States
(12) Patent Application Publication
(10) Pub. No.: US 2004/0019000 A1
(43) Pub. Date: Jan. 29, 2004

(54) Polyalkyleneamine-containing oligomers

(76) Inventors: Muthiah Manoharan, Carlsbad, CA (US); Andrei P. Guzaev, Carlsbad, CA (US); Martin A. Maier, Carlsbad, CA (US)

Correspondence Address: WOODCOCK WASHBURN LLP, ONE LIBERTY PLACE - 46TH FLOOR, PHILADELPHIA, PA, 19103, US

(21) Appl. No.: 10/199,585

(22) Filed: Jul. 19, 2002

(51) Int. Cl. A61K 48/00; C07H 21/02; C07H 21/04; C08G 63/48; C08G 63/91

(52) U.S. Cl. 514/44; 525/54.2; 536/23.1

(57) ABSTRACT

The present invention relates to novel polyethylenamine-conjugated oligomeric compounds and to methods of making such compounds. The invention further relates to methods of enhancing the cellular uptake of oligomeric compounds comprising conjugating the compounds to fusogenic moieties such as polyethylenimine.

### FIELD OF THE INVENTION

The present invention is directed to polyalkyleneamine-conjugated oligomeric compounds and to methods of making and using such compounds.

### BACKGROUND OF THE INVENTION

Nearly all disease states in multicellular organisms involve the action of proteins. Classic therapeutic approaches have focused on the interaction of proteins with other molecules in efforts to moderate the proteins' disease-causing or disease-potentiating activities. In newer therapeutic approaches, modulation of the production of proteins has been sought. A general object of some current therapeutic approaches is to interfere with or otherwise modulate gene expression.

One method for inhibiting the expression of specific genes involves the use of oligonucleotides, particularly oligonucleotides that are complementary to a specific target messenger RNA (mRNA) sequence, known as antisense oligonucleotides. Several oligonucleotides are currently undergoing clinical trials for such use. Phosphorothioate antisense oligonucleotides are presently being used as antiviral agents in human clinical trials.

Oligonucleotides and their analogs can be designed to have particular properties. A number of chemical modifications have been introduced into oligomeric compounds to increase their usefulness as therapeutic agents. Such modifications include those designed to increase binding affinity to a target strand, to increase cell penetration, to stabilize against nucleases and other enzymes that degrade or interfere with the structure or activity of the oligonucleotide, to provide a mode of disruption (terminating event) once the oligonucleotide is bound to a target, and to improve the pharmacokinetic properties of the oligonucleotide. Despite such modifications, the cellular uptake of oligomeric compounds remains poor.

Oligonucleotides have been formulated with various with transfection agents, including anionic and cationic lipids and polyamines, in an attempt to improve their ability to permeate biological membranes. Dheur, S.; Saison-Behmoaras, T. E. Methods Enzymol. 2000, 313, 56-73; Vinogradov, S.; Batrakova, E.; Kabanov, A. V. Polym. Prepr. (Am. Chem. Soc., Div. Polym. Chem.) 2000, 41, 1641-1642; Vinogradov, S.; Batrakova, E.; Kabanov, A. Colloids Surf., B 1999, 16, 291-304; Bandyopadhyay, P.; Ma, X.; Linehan-Stieers, C.; Kren, B. T.; Steer, C. J. J. Biol. Chem. 1999, 274, 10163-10172; Auvray, P.; Sourdaine, P.; Seralini, G. E. Biochem. Biophys. Res. Commun. 1998, 253, 1-9; Demeneix, B. A.; Boussif, O.; Zanta, M. A.; Remy, J. S.; Behr, J. P. Nucleosides Nucleotides 1997, 16, 1121-1127. Of the transfection agents used, polyethylenimines (PEI) are the most efficient and least expensive delivery vehicles. Kren, B. T.; Parashar, B.; Bandyopadhyay, P.; Chowdhury, N. R.; Chowdhury, J. R.; Steer, C. J. Proc. Natl. Acad. Sci. U.S.A. 1999, 96, 10349-10354. It was observed, however, that, although complexes of excess PEI and oligonucleotide phosphorothioates were efficiently taken up by cells, the oligonucleotides failed to dissociate in the cytoplasm, resulting in no enhancement in the antisense activity of the oligonucleotides. Dheur, S.; Dias, N.; Van Aerschot, A.; Herdewijn, P.; Bettinger, T.; Remy, J. -S.; Helene, C.; Saison-Behmoaras, E. T. Antisense Nucleic Acid Drug Dev. 1999, 9, 515-525. Nuclear localization of antisense oligonucleotides upon cellular delivery is essential for their activity.

A need therefore exists in the art for the development of means to improve the cellular uptake and cellular distribution of oligomeric compounds.

### SUMMARY OF THE INVENTION

In certain embodiments, this invention relates to oligomeric compounds of formula I:

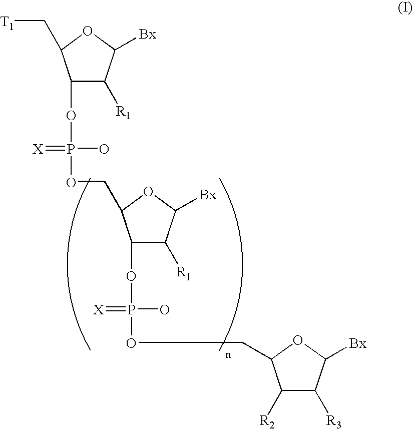

wherein:

T₁ is hydroxyl or a protected hydroxyl;

each Bx is an optionally protected heterocyclic base moiety;

each R₁ is, independently, hydrogen or a sugar substituent group;

each X is, independently, S or O;

n is from 2 to about 50;

one of R₂ and R₃ is -L-R₄, and the other of R₂ and R₃ is -L-R₄, hydrogen or a sugar substituent group;

each L is a linking group; and

R₄ is a polyalkyleneamino radical having a molecular weight of from about 100 daltons to about 100,000 daltons.

In other embodiments, the invention relates to oligomeric compounds of formula VI:

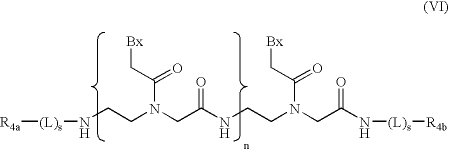

wherein:

each Bx is an optionally protected heterocyclic base moiety;

n is from 2 to about 50;

each L is a linking group;

each s is 0 or 1;

at least one of R₄ₐ and R_{4b} is a polyethylenamino radical having a molecular weight of from about 100 daltons to about 100,000 daltons, and if R₄ₐ or R_{4b} is not a polyethylenamino radical it is hydrogen, an amino protecting group, a carbonyl protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid, or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl.

In further embodiments, the invention relates to compounds comprising an oligomeric moiety, a fusogenic moiety, and a targeting moiety.

In other embodiments, the invention relates to methods of enhancing the cellular uptake of an oligomeric compound comprising conjugating the compound to a fusogenic moiety. In additional embodiments, the invention relates to methods of enhancing the cellular uptake of an oligomeric compound comprising conjugating the compound to a fusogenic moiety, and further comprising conjugating the oligomeric compound-fusogenic moiety conjugate to a targeting moiety.

### BRIEF DESCRIPTION OF THE DRAWINGS

FIG. 1: Uptake of survivin-PEI conjugates by Jurkat cells after 24 hours.

FIG. 2: Viability of Jurkat cells after 24 hours of incubation with the compounds, as determined by exclusion of propidium iodide.

### DETAILED DESCRIPTION OF THE PREFERRED EMBODIMENTS

In the context of this invention, the terms “oligomer” and “oligomeric compound” refer to a plurality of naturally-occurring or non-naturally-occurring nucleosides joined together in a specific sequence. The terms “oligomer” and “oligomeric compound” include oligonucleotides, oligonucleotide analogs, oligonucleosides and chimeric oligomeric compounds where there are more than one type of internucleoside linkages dividing the oligomeric compound into regions. Oligomeric compounds are typically structurally distinguishable from, yet functionally interchangeable with, naturally-occurring or synthetic wild-type oligonucleotides. Thus, oligomeric compounds include all such structures that function effectively to mimic the structure and/or function of a desired RNA or DNA strand, for example, by hybridizing to a target.

Oligomeric compounds according to the present invention preferably comprise from about 5 to about 50 monomer subunits and, hence, about 5 to about 50 nucleosidic bases. It is more preferred that such compounds comprise from about 8 to about 30 monomer subunits, with 15 to 25 monomer subunits being particularly preferred.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for nucleic acid target and increased stability in the presence of nucleases.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure. However, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

The present invention provides oligomeric compounds comprising a plurality of linked nucleosides wherein the preferred internucleoside linkage is a 3′,5′-linkage. Alternatively, 2′,5′-linkages can be used (as described in U.S. application Ser. No. 09/115,043, filed Jul. 14, 1998). A 2′,5′-linkage is one that covalently connects the 2′-position of the sugar portion of one nucleotide subunit with the 5′-position of the sugar portion of an adjacent nucleotide subunit.

Specific examples of preferred oligomeric compounds useful in this invention include those having modified backbones or non-naturally occurring internucleoside linkages. As defined in this specification, modified backbones include those having a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

As used herein, the term “oligomeric compound conjugate” refers to an oligomeric compound to which one or more chemical entities are covalently attached. In preferred embodiments of the invention, an oligomeric compound is conjugated to a polyalkyleneamino radical. In particularly preferred embodiments of the invention, an oligomeric compound is conjugated to a polyethyleneamino radical.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′,5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative Phosphorus Containing Linkages

phosphorodithioate (—O—P(S)(S)—O—);

phosphorothioate (—O—P(S)(O)—O—);

phosphoramidate (—O—P(O)(NJ₂)—O—);

phosphonate (—O—P(J)(O)—O—);

phosphotriesters (—O—P(OJ)(O)—O—);

phophosphoramidate (—O—P(O)(NJ)—S—);

thionoalkylphosphonate (—O—P(S)(J)—O—);

thionoalkylphosphotriester (—O—P(O)(OJ)—S—);

phosphoramidate (—N(J)—P(O)(O)—O—);

boranophosphate (—R⁵—P(O)(O)—J—);

where J denotes a substituent group which is commonly hydrogen or an alkyl group or a more complicated group that varies from one type of linkage to another.

Representative United States patents that teach the preparation of the above phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative Non-Phosphorus Containing Linkages

thiodiester (—O—C(O)—S—);

thionocarbamate (—O—C(O)(NJ)—S—);

siloxane (—O—Si(J)₂—O—);

carbamate (—O—C(O)—NH— and —NH—C(O)—O—)

sulfamate (—O—S(O)(O)—N— and —N—S(O)(O)—N—;

morpholino sulfamide (—O—S(O)(N(morpholino)-);

sulfonamide (—O—SO₂—NH—);

sulfide (—CH₂—S—CH₂—);

sulfonate (—O—SO₂—CH₂—);

N,N′-dimethylhydrazine (—CH₂—N(CH₃)—N(CH₃)—);

thioformacetal (—S—CH₂—O—);

formacetal (—O—CH₂—O—);

thioketal (—S—C(J)₂—O—); and

ketal (—O—C(J)₂—O—);

amine (—NH—CH₂—CH₂—);

hydroxylamine (—CH₂—N(J)—O—);

hydroxylimine (—CH═N—O—); and

hydrazinyl (—CH₂—N(H)—N(H)—).

where J denotes a substituent group which is commonly hydrogen or an alkyl group or a more complicated group that varies from one type of linkage to another.

Representative United States patents that teach the preparation of the above oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Particularly preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

In certain preferred oligonucleotide mimetics, both the sugar and the internucleoside linkage, i.e., the backbone, of the nucleotide units are replaced with other groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). Peptide nucleic acids (PNAs) are DNA analogs that contain an uncharged pseudo-peptide backbone with N-(2-aminoethyl)glycine units to which the nucleobases are attached via methylene carbonyl linkers, and are described in Nielsen, P. E., Egholm, M., Berg, R. H., and Buchardt, O. (1991) Science 254, 1497-500; Egholm, M., Buchardt, O., Nielsen, P. E., and Berg, R. H. (1992) J. Am. Chem. Soc. 114, 1895-7; Egholm, M., Buchardt, O., Christensen, L., Behrens, C., Freier, S. M., Driver, D. A., Berg, R. H., Kim, S. K., Norden, B., and Nielsen, P. E. (1993) Nature (London) 365, 566-8; and Dueholm, K. L., and Nielsen, P. E. (1997) New J. Chem. 21, 19-31. PNA is capable of sequence-specific recognition of DNA and RNA obeying the Watson-Crick hydrogen bonding scheme, and the hybrid complexes exhibit extraordinary thermal stability and unique ionic strength effects. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 1991, 254, 1497-1500.

PNA has been used for many therapeutic and genetic applications, including monitoring telomere length, screening for genetic mutations, affinity capture of nucleic acids, and antisense-mediated target reduction. Such applications are described in Corey, D. R. (1997) in Trends Biotechnol. pp 224-229; Lansdorp, P. M., Verwoerd, N. P., van de Rijke, F. M., Dragowska, V., Little, M. -T., Dirks, R. W., Raap, A. K., and Tanke, H. J. (1996) Hum. Mol. Genet. 5, 685-691; Orum, H., Nielsen, P. E., Egholm, M., Berg, R. H., Buchardt, O., and Stanley, C. (1993) Nucleic Acids Research 21, 5332-6; Carlsson, C., Jonsson, M., Norden, B., Dulay, M. T., Zare, R. N., Noolandi, J., Nielsen, P. E., Tsui, L. -C., and Zielenski, J. (1996) in Nature (London) pp 207; Bukanov, N. O., Demidov, V. V., Nielsen, P. E., and Frank-Kamenetskii, M. D. (1998) Proceedings of the National Academy of Sciences of the United States of America 95, 5516-20; and Norton, J. C., Piatyszek, M. A., Wright, W. E., Shay, J. W., and Corey, D. R. (1996) Nat. Biotechnol. 14, 615-19.

Besides binding target mRNA with high affinity, PNAs are highly resistant to nuclease and protease degradation, and display mismatch sequence discrimination, thus making them interesting third generation antisense molecules with potential therapeutic application. Such properties are described in Norton, J. C., Piatyszek, M. A., Wright, W. E., Shay, J. W., and Corey, D. R. (1996) Nat. Biotechnol. 14, 615-19; and Egholm, M., Buchardt, O., Christensen, L., Behrens, C., Freier, S. M., Driver, D. A., Berg, R. H., Kim, S. K., Norden, B., and Nielsen, P. E. (1993) Nature (London) 365, 566-8.

PNA oligomers have demonstrated in vitro transcriptional and translational block of many genes, as described in Mologni, L., Nielsen, P. E., and Gambacorti-Passerini, C. (1999) Biochem. Biophys. Res. Commun. 264, 537-543. Furthermore, PNA oligomers have been shown to induce triplex mediated mutagenesis of a chromosomal gene in mouse cells, as described in Faruoi, A. F., Egholm, M., and Glazer, P. M. (1998) Proc. Natl. Acad. Sci. U.S.A. 95, 1398-1403.

A heterocyclic base moiety (often referred to in the art simply as a “base” or a “nucleobase”) amenable to the present invention includes both naturally and non-naturally occurring nucleobases. The heterocyclic base moiety further may be protected wherein one or more functionalities of the base bears a protecting group. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993.

Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

In one aspect of the present invention oligomeric compounds are prepared having polycyclic heterocyclic compounds in place of one or more heterocyclic base moieties. A number of tricyclic heterocyclic comounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

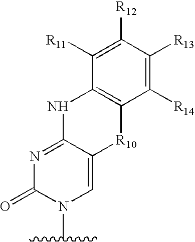

Representative cytosine analogs that make 3 hydrogen bonds with a guanosine in a second strand include 1,3-diazaphenoxazine-2-one (R_{10═}O, R₁₁-R₁₄═H) [Kurchavov, et al., Nucleosides and Nucleotides, 1997, 16, 1837-1846], 1,3-diazaphenothiazine-2-one (R₁₀═S, R₁₁-R₁₄═H), [Lin, K. -Y.; Jones, R. J.; Matteucci, M. J. Am. Chem. Soc. 1995, 117, 3873-3874] and 6,7,8,9-tetrafluoro-1,3-diazaphenoxazine-2-one (R₁₀═O, R₁₁-R₁₄═F) [Wang, J.; Lin, K. -Y., Matteucci, M. Tetrahedron Lett. 1998, 39, 8385-8388]. Incorporated into oligonucleotides these base modifications were shown to hybridize with complementary guanine and the latter was also shown to hybridize with adenine and to enhance helical thermal stability by extended stacking interactions.

Further helix-stabilizing properties have been observed when a cytosine analog/substitute has an aminoethoxy moiety attached to the rigid 1,3-diazaphenoxazine-2-one scaffold (R_{10═}O, R₁₁═—O—(CH₂)₂—NH₂, R₁₂₋₁₄═H ) [Lin, K. -Y.; Matteucci, M. J. Am. Chem. Soc. 1998, 120, 8531-8532]. Binding studies demonstrated that a single incorporation could enhance the binding affinity of a model oligonucleotide to its complementary target DNA or RNA with a ΔTₘ of up to 18° relative to 5-methyl cytosine (dC5^{me}), which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. No. 6,028,183, which issued on May 22, 2000, and U.S. Pat. No. 6,007,992, which issued on Dec. 28, 1999, the contents of both are commonly assigned with this application and are incorporated herein in their entirety. Such compounds include those having the formula:

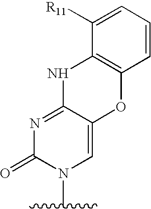

wherein R₁₁ includes (CH₃)₂N—(CH₂)₂—O—; H₂N—(CH₂)₃—; Ph—CH₂—O—C(═O)—N(H)—(CH₂)₃—; H₂N—; Fluorenyl-CH₂—O—C(═O)—N(H)—(CH₂)₃—; Phthalimidyl-CH₂—O—C(═O)—N(H)—(CH₂)₃—; Ph—CH₂—O—C(═O)—N(H)—(CH₂)₂—O—; Ph—CH₂—O—C(═O)—N(H)—(CH₂)₃—O—; (CH₃)₂N—N(H)—(CH₂)₂—O—; Fluorenyl-CH₂—O—C(═O)—N(H)—(CH₂)₂—O—; Fluorenyl—CH₂—O—C(═O)—N(H)—(CH₂)₃—O—; H₂N—(CH₂)₂—O—CH₂—; N₃—(CH₂)₂—O—CH₂—; H₂N—(CH₂)₂—O—, and NH₂C(═NH)NH—.

Also disclosed are tricyclic heterocyclic compounds of the formula:

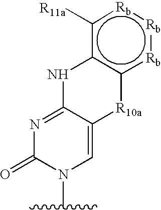

wherein

R₁₀ₐ is O, S or N—CH₃;

R₁₁ₐ is A(Z)ₓ₁, wherein A is a spacer and Z independently is a label bonding group bonding group optionally bonded to a detectable label, but R₁₁ₐ is not amine, protected amine, nitro or cyano;

X1 is 1, 2or3; and

Ris independently —CH═, —N═, —C(C₁₋₈ alkyl)═ or —C(halogen)═, but no adjacent R_{b} are both —N═, or two adjacent R_{b} are taken together to form a ring having the structure:

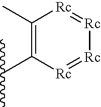

where R_{c} is independently —CH═, —N═, —C(C₁₋₈ alkyl)═ or —C(halogen)═, but no adjacent R_{b} are both —N═.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity [Lin, K. -Y.; Matteucci, M. J. Am. Chem. Soc. 1998, 120, 8531-8532]. The activity enhancement was even more pronounced in case of G-clamp, as a single substitution was shown to significantly improve the in vitro potency of a 20 mer 2′-deoxyphosphorothioate oligonucleotides [Flanagan, W. M.; Wolf, J. J.; Olson, P.; Grant, D.; Lin, K. -Y.; Wagner, R. W.; Matteucci, M. Proc. Natl. Acad. Sci. USA, 1999, 96, 3513-3518]. Nevertheless, to optimize oligonucleotide design and to better understand the impact of these heterocyclic modifications on the biological activity, it is important to evaluate their effect on the nuclease stability of the oligomers.

Further tricyclic and tetracyclic heteroaryl compounds amenable to the present invention include those having the formulas:

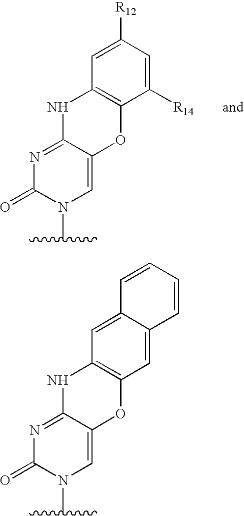

wherein R₁₄ is NO₂ or both R₁₄ and R₁₂ are independently —CH₃. The synthesis of these compounds is dicslosed in U.S. Pat. No. 5,434,257, which issued on Jul. 18, 1995, U.S. Pat. No. 5,502,177, which issued on Mar. 26, 1996, and U.S. Pat. No. 5,646, 269, which issued on Jul. 8, 1997, the contents of which are commonly assigned with this application and are incorporated herein in their entirety.

Further polycyclic heterocyclic base moieties having the formula:

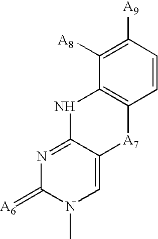

wherein:

A₆ is O or S;

A₇ is CH₂, N—CH₃, O or S;

each A₈ and A₉ is hydrogen or one of A₈ and A₉ is hydrogen and the other of A₈ and A₉ is selected from the group consisting of:

—O—(CH₂)ₚ₁-G and

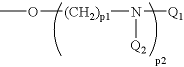

wherein:

wherein:

G is —CN, —OA₁₀, —SA₁₀, —N(H)A₁₀, —ON(H)A₁₀ or —C(═NH)N(H)A₁₀;

Q₁ is H, —NHA₁₀, —C(═O)N(H)A₁₀, —C(═S)N(H)A₁₀ or —C(═NH)N(H)A₁₀;

each Q₂ is, independently, H or Pg;

A₁₀ is H, Pg, substituted or unsubstituted C₁-C₁₀ alkyl, acetyl, benzyl, —(CH₂)ₚ₃NH₂, —(CH₂)ₚ₃N(H)Pg, a D or L α-amino acid, or a peptide derived from D, L or racemic α-amino acids;

Pg is a nitrogen, oxygen or thiol protecting group;

each p1 is, independently, from 2 to about 6;

p2 is from 1 to about 3; and

p3 is from 1 to about 4;

are disclosed in U.S. patent application Ser. No. 09/996,292 filed Nov. 28, 2001, which is commonly owned with the instant application, and is herein incorporated by reference.

The compounds described herein may have asymmetric centers. Unless otherwise indicated, all chiral, diastereomeric, and racemic forms are included in the present invention. Geometric isomers may also be present in the compounds described herein, and all such stable isomers are contemplated by the present invention. It will be appreciated that compounds in accordance with the present invention that contain asymmetrically substituted carbon atoms may be isolated in optically active or racemic forms or by synthesis.

The present invention includes all isotopes of atoms occurring in the intermediates or final compounds. Isotopes include those atoms having the same atomic number but different mass numbers. By way of example, and without limitation, isotopes of hydrogen include tritium and deuterium.

As used herein, the term “sugar substituent group” refers to a group that is covalently attached to an oligomeric compounds. The 2′-position of oligomeric compounds has been a preferred position for covalent attachment of sugar substituent groups. However, the 3′ and 5′-positions and the heterocyclic base moiety of selected nucleosides have also been modified with sugar substituent groups.

Preferred sugar substituent groups include OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred sugar substituent groups include: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred sugar substituent group includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred sugar substituent group includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other preferred sugar substituent groups include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligomeric compounds may also have sugar mimetics such as cyclobutyl moieties in place of the pentofuranosyl sugar. Representative United States patents that teach the preparation of such modified sugar structures include, but are not limited to, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

It is not necessary for all positions in a given oligomeric compound to be uniformly modified, and in fact more than one of the aforementioned modifications may be incorporated in a single compound or even at a single nucleoside within an oligonucleotide.

The present invention also includes oligomeric compounds that are chimeric compounds. “Chimeric” oligomeric compounds or “chimeras,” in the context of this invention, are oligomeric compounds, particularly oligonucleotides, that contain two or more chemically distinct regions, each made up of at least one monomer unit, i.e., a nucleotide in the case of an oligonucleotide compound. Chimeric oligonucleotides typically contain at least one region wherein the oligonucleotide is modified so as to confer increased resistance to nuclease degradation, increased cellular uptake, and/or increased binding affinity for the target nucleic acid upon the oligonucleotide. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

In certain embodiments, the oligomeric compounds of the invention can be chimeric oligonucleotides, including “gapmers,” “inverted gapmers,” or “hemimers.” In a “hemimer,” a single terminal (either 5′ or 3′) region of the oligonucleotide contains modified nucleosides. When both termini of the oligonucleotide contain modified nucleosides, the oligonucleotide is called a “gapmer” and the modified 5′- and 3′-terminal regions are referred to as “wings”. In a gapmer, the 5′ and 3′ wings can contain nucleosides modified in the same or different manner. In an “inverted gapmer” a central region of the oligonucleotide contains modified nucleosides.

At the intracellular level a major barrier to the successful delivery of antisense oligomeric compounds involves endosomal entrapment of oligomeric drugs. Several strategies to increase the efficiency of cytoplasmic delivery of oligonucleotides exist. Methods that cause endosomal release of oligomeric drug compounds are critical to successful delivery of drug molecules. Cationic polymers such as polyethyleneimines cause endosomal escape, which occurs by a proton sponge mechanism. The cationic polymers have amine groups with a pKa in the range of 4.5 to 5.5. Outside the cell and endosomal compartments, the polymers behave as neutral molecules. Once inside endosomes, the polymers are protonated and the endosome is effectively buffered. Changing the pH of endosomes increases the osmotic pressure, ultimately leading to swelling, rupture, and release of the endosomal contents into the cytoplasm of the cell.

Cationic polymers capable of acting as fusogenic groups include lipophilic polyamines, polyethylenimines, and polyallylamines. Other agents capable of acting as fusogenic groups include fusogenic peptides, oligomeric imidazoles, histidines, pyridines, hydroxylamines, substituted hydroxylamines, hydrazines, substituted hydrazines, thioureas (e.g. dithiobiur) and imines. In each molecule, the nitrogen atom has a pKa in the range of 5.0.

Chloroquine is thought to have buffering capacity that prevents endosomal acidification and has been shown to enhance the transfection activity of polycation/DNA complexes. The use of chloroquine is limited to in vitro applications since the concentration of chloroquine required to enhance transfection is likely to be toxic in vivo.

Many viruses use membrane destabilizing proteins (fusogenic peptides) to promote endosomal release, and one of the best studied of these systems is the influenza virus haemaglutinnin. The fusion domain of this protein is located at the N-terminus of subunit HA 2 and the peptide sequence, and modifications of it, have been shown to significantly enhance transfection efficiency in a number of polymer/DNA systems. Fusogenic properties of the C-terminal domain of the Alzheimer beta-amyloid peptide have been reported (see Pillot T; et al. J Biol Chem, 271(46):28757-65). The amyloid 29-42 peptide is the most potent fusogenic peptide.

As used herein, the term “fusogenic moiety” or “fusogenic compound” refers to any agent that enhances the release of a molecule of interest from an endosome.

In certain embodiments, the novel compounds of the invention comprise oligomeric compounds conjugated to one or more fusogenic moieties. In some embodiments of the invention, the one or more fusogenic moieties are polyalkyleneamino radicals.

In certain embodiments of the invention, the fusogenic moiety is a polyalkyleneamino radical of formula VII:

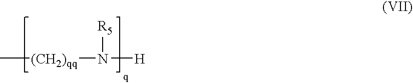

wherein qq is from 1 to about 10; q is from about 2 to about 1700; and each R₅ is, independently, H or a group of formula VIII:

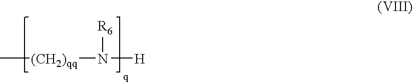

wherein p is from 1 to about 1000, and each R₆ is, independently, H or a group of formula VII.

In certain embodiments of the invention, the fusogenic moiety is a polyethyleneamino radical having a molecular weight of from about 100 to about 100,000. In preferred embodiments of the invention, the fusogenic moiety is a polyethyleneamino radical having a molecular weight of from about 200 to about 40,000. In more preferred embodiments of the invention, the fusogenic moiety is a polyethyleneamino radical having a molecular weight of from about 600 to about 20,000. In particularly preferred embodiments of the invention, the fusogenic moiety is a polyethylenamino radical of formula II:

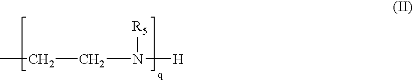

wherein q is from about 2 to about 1700; and each R₅ is, independently, H or a group of formula III:

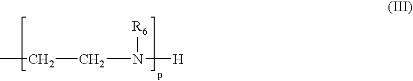

wherein p is from 1 to about 1000; and each R₆ is, independently, H or a group of formula (II).

In certain embodiments of the invention, the polyethyleneamino radical is a radical of formula II and each R₅ is H. In other embodiments of the invention, the polyethyleneamino radical is a radical of formula II and at least one R₅ is a group of formula III.

In preferred embodiments of the invention, the polyethyleneamino radical is a radical of formula II and each L is, independently, a linking group of formula IV:

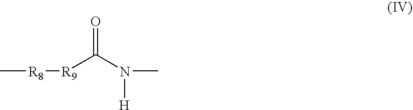

wherein: R₈ is —O—, phosphate or phosphorothioate and is covalently attached to the R₂ or R₃ position of formula I; R₉ is (CH₂)ₘ, (CH₂)ₘₘ—C₆-C₂₀ aryl or a polyethylene glycol—(CH₂)₂—[O—(CH₂)₂]ₘₘₘ—; m is from 1 to about 6; mm is from 1 to about 6; and mmm is from 1 to about 6. Further in the context of this specification, aryl groups (generally C₆-C₂₀) include but are not limited to substituted and unsubstituted aromatic hydrocarbyl groups. Aralkyl groups (generally C₇-C₂₀) include but are not limited to groups having both aryl and alkyl functionalities, such as benzyl and xylyl groups. Preferred aryl and aralkyl groups include, but are not limited to, phenyl, benzyl, xylyl, naphthyl, toluyl, pyrenyl, anthracyl, azulyl, phenethyl, cinnamyl, benzhydryl, and mesityl. Typical substituents for substitution include, but are not limited to, hydroxyl, alkoxy, alcohol, benzyl, phenyl, nitro, thiol, thioalkoxy, halogen, or alkyl, aryl, alkenyl, or alkynyl groups.

In particularly preferred embodiments of the invention, the polyethyleneamino radical is a radical of formula II and at least one L is a group of formula V:

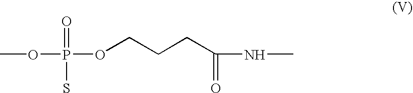

In certain embodiments of the invention, the polyethylenamino radical is a radical of formula II and R₃ is -L-R₄.

In other embodiments, the novel compounds of the invention comprise an oligomeric moiety, one or more fusogenic moieties, and one or more targeting moieties. As used herein, the term “targeting moiety” or “targeting compound” refers to any agent that directs a molecule of interest to particular cells or particular types of cells. Examples of targeting moieties include, but are not limited to, ligands that bind to cellular receptors, such as, for example, transferrin, folate, epidermal growth factor, nerve growth factor, and insulin. Targeting moieties also include, but are not limited to, alpha-fetoprotein, galactose, galactosamine, lactose, mannose, polyclonal antibodies, moloclonal antibodies, Vitamin B₁₂, ibuprofen, cholesterol, low-density lipoprotein, and peptides comprising an arginine-glycine-aspartic acid sequence.

In particular embodiments, a fusogenic moiety is covalently linked to the oligomeric moiety and a targeting moiety is covalently linked to the oligomeric moiety. In other embodiments of the invention, a fusogenic moiety covalently linked to the oligomeric moiety and a targeting moiety covalently linked to the fusogenic moiety.

Although not wishing to be bound by any theory, it is thought that conjugation of oligomeric compounds to one or more fusogenic moieties enhances the pharmacodynamic and pharmacokinetic properties of the compounds by improving the ability of the compounds to penetrate cell membranes and by improving the cellular distribution of the compounds once the compounds are inside cells. In addition, it is thought that conjugation of oligomeric compounds to one or more targeting moieties results in uptake of the oligomeric compound conjugates by specific types of cells. For example, targeting moieties can be ligands for cell surface receptors that are expressed by certain specific types of cells. Conjugation of an oligomeric compound to such a ligand and administration of the conjugate to an organism is thought to result in uptake of the conjugate by cells expressing cell surface receptors that bind the ligand.

In certain embodiments, the present invention relates to methods of enhancing the cellular uptake of oligomeric compounds comprising conjugating the compounds to one or more fusogenic moieties. In other embodiments, the present invention relates to methods of enhancing the cellular uptake of oligomeric compounds comprising conjugating the compounds to one or more fusogenic moieties and to one or more targeting moieties.

In particular embodiments of the invention, polyalkyleneamine-conjugated oligomeric compounds are prepared by assembling oligomeric compounds on support media, and conjugating the support-bound oligomeric compounds to polyalkyleneamines or polyalkyleneimies. In preferred embodiments of the invention, polyethyleneamine-conjugated oligomeric compounds are prepared by assembling oligomeric compounds on support media derivatized with a reactive thioester group. An amine of a polyethyleneamine or polyethyleneimie compound is then reacted with the thioester group, and the thioester is converted to a carboxamido group, resulting in conjugation of the oligomeric compound to the polyethyleneamine group and release of the oligomeric compound from the solid support.

In certain embodiments of the invention, polyethyleneamine-conjugated oligomeric compounds are prepared using derivatized support media. In preferred embodiments of the invention, the support media is derivatized with a thioester group. In preferred embodiments of the invention, the support media is synthesized by treating long chain alkylamine controlled pore glass with 2,2′-dithiodiglycolic acid, N,N′-diisopropylcarbodiimide, and 4-dimethylaminopyridine (DMAP, 0.1 equiv.) in pyridine. The product is capped with Ac₂O/N-methylimidazole/Py/THF, and the disulfide bond is reduced with 1,4-dithiothreitol (DTT) in aqueous MeCN. The solid support is treated with 4-(4,4′-dimethoxytrityloxy)butyric acid and capped with Ac₂O/N-methylimidazole/Py/THF to yield the derivatized solid support.

In preferred embodiments of the invention, oligomeric compounds are synthesized on support media derivatized with a thioester group according to standard oligonucleotide synthesis procedures. In some embodiments of the invention, the support-bound oligonucleotides are then treated with polyethyleneamine, spermine, and thiophenol in aqueous MeCN to yield polyethyleneamine-conjugated oligomeric compounds. In other embodiments of the invention, the support-bound oligonucleotides are treated with polyethyleneimines and thiophenol, the reaction mixture is diluted with concentrated aqueous ammonium hydroxide, and the solution is heated and evaporated. The residue is dissolved in water and neutralized with aqueous AcOH, resulting in precipitation of oligonucltotide conjugates complexed with excess polyethyleneimine. The precipitate is washed with MeCN and ether and re-dissolved in a mixture of piperidine and DMSO. The polyethyleneamine-conjugated oligomeric compounds are then purified on a Sephadex G25 column, and then further purified by reverse-phase HPLC.

Standard procedures for the synthesis of oligomeric compounds involve attachment of a first nucleoside or larger nucleosidic synthon to support media followed by iterative elongation of the nucleoside or nucleosidic synthon to yield a final oligomeric compound. In some embodiments of the invention, oligomeric compounds are synthesized by attaching a 5′-O-protected nucleoside to a solid support derivatized with a thioester group, deprotecting the 5′-hydroxyl of the nucleoside with a deprotecting reagent, reacting the deprotected 5 ′-hydroxyl with a 5 ′-protected activated phosphorus compound to produce a covalent linkage therebetween, oxidizing or sulfurizing the covalent linkage, and repeating the deprotecting, reacting, and oxidizing steps to produce an oligomer attached to the derivatized support media.

Support media can be selected to be insoluble or to have variable solubility in different solvents, which allows the growing oligomer to be kept out of or in solution as desired. Traditional solid supports are insoluble, while soluble supports have recently been introduced. Soluble polymer supports allow the bound oligomer to be precipitated or dissolved at desired points in the synthesis (Gravert et al., Chem. Rev., 1997, 97, 489-510).

Representative support media amenable to the present invention include, without limitation, controlled pore glass (CPG); oxalyl-controlled pore glass (see, e.g., Alul, et al., Nucleic Acids Research 1991, 19, 1527); TENTAGEL Support, (see, e.g., Wright, et al., Tetrahedron Letters 1993, 34, 3373); and POROS, a copolymer of polystyrene/divinylbenzene available from Perceptive Biosystems. Use of poly(ethylene glycol) of molecular weight between 5 and 20 kDa as a soluble support media for large-scale synthesis of phosphorothioate oligonucleotides is described in Bonora et al., Organic Process Research & Development, 2000, 4, 225-231. Equipment for such synthesis is sold by several vendors including, for example, Applied Biosystems (Foster City, Calif.).

Other means for synthesis of oligomeric compounds may additionally or alternatively be employed. Techniques for synthesizing oligonucleotides, such as phosphorothioates and alkylated derivatives, are familiar to those of ordinary skill in the art.

Activated phosphorus compositions (e.g. compounds having activated phosphorus-containing substituent groups) may be used in coupling reactions for the synthesis of oligomeric compounds. As used herein, the term “activated phosphorus composition” includes monomers and oligomers that have an activated phosphorus-containing substituent group that reacts with a hydroxyl group of another monomeric or oligomeric compound to form a phosphorus-containing internucleotide linkage. Such activated phosphorus groups contain activated phosphorus atoms in P^{III} valence state. Such activated phosphorus atoms are known in the art and include, but are not limited to, phosphoramidite, H-phosphonate, phosphate triesters and chiral auxiliaries. A preferred synthetic solid phase synthesis utilizes phosphoramidites as activated phosphates. The phosphoramidites utilize P^{III} chemistry. The intermediate phosphite compounds are subsequently oxidized to the P^{V} state using known methods to yield, in a preferred embodiment, phosphodiester or phosphorothioate internucleotide linkages. Additional activated phosphates and phosphites are disclosed in Tetrahedron Report Number 309 (Beaucage and Iyer, Tetrahedron, 1992, 48, 2223-2311).

A representative list of activated phosphorus-containing monomers or oligomers include those having the formula:

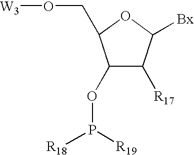

wherein

each Bx is, independently, a heterocyclic base moiety or a blocked heterocyclic base moiety; and

each R₁₇ is, independently, H, a blocked hydroxyl group, a sugar substituent group, or a blocked substituent group;

W₃ is an hydroxyl protecting group, a nucleoside, a nucleotide, an oligonucleoside or an oligonucleotide;

R₁₈ is N(L₁)L₂,

each L₁ and L₂ is, independently, C₁₋₆ alkyl;

or L₁ and L₂ are joined together to form a 4- to 7-membered heterocyclic ring system including the nitrogen atom to which L₁ and L₂ are attached, wherein said ring system optionally includes at least one additional heteroatom selected from O, N and S; and

R₁₉ is X₁;

X₁ is Pg-O—, Pg-S—, C₁-C₁₀ straight or branched chain alkyl, CH₃(CH₂)ₚ₅—O— or R₂₀R₂₁N—;

p5 is from 0 to 10;

Pg is a protecting group;

each R₂₀ and R₂₁ is, independently, hydrogen, C₁-C₁₀ alkyl, cycloalkyl or aryl;

or optionally, R₂₀ and R₂₁, together with the nitrogen atom to which they are attached form a cyclic moiety that may include an additional heteroatom selected from O, S and N; or

R₁₈ and R₁₉ together with the phosphorus atom to which R₁₈ and R₁₉ are attached form a chiral auxiliary.

Groups attached to the phosphorus atom of internucleotide linkages before and after oxidation (R₁₈ and R₁₉) can include nitrogen containing cyclic moieties such as morpholine. Such oxidized internucleoside linkages include a phosphoromorpholidothioate linkage (Wilk et al., Nucleosides and nucleotides, 1991, 10, 319-322). Further cyclic moieties amenable to the present invention include mono-, bi- or tricyclic ring moieties that may be substituted with groups such as oxo, acyl, alkoxy, alkoxycarbonyl, alkyl, alkenyl, alkynyl, amino, amido, azido, aryl, heteroaryl, carboxylic acid, cyano, guanidino, halo, haloalkyl, haloalkoxy, hydrazino, ODMT, alkylsulfonyl, nitro, sulfide, sulfone, sulfonamide, thiol and thioalkoxy. A preferred bicyclic ring structure that includes nitrogen is phthalimido.

In the context of this specification, alkyl (generally C1-C20), alkenyl (generally C2-C20), and alkynyl (generally C2-C20) groups include, but are not limited to, substituted and unsubstituted straight chain, branch chain, and alicyclic hydrocarbons, including methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl and other higher carbon alkyl groups. Further examples include 2-methylpropyl, 2-methyl-4-ethylbutyl, 2,4-diethylbutyl, 3-propylbutyl, 2,8-dibutyldecyl, 6,6-dimethyl-oxtyl, 6-propyl-6-butyloctyl, 2-methylbutyl, 2-methylpentyl, 3-methylpentyl, 2-ethylhexyl and other branched chain groups, allyl, crotyl, propargyl, 2-pentenyl and other unsaturated groups containing a pi bond, cyclohexane, cyclopentane, adamantane as well as other alicyclic groups, 3-penten-2-one, 3-methyl-2-butanol, 2-cyanooctyl, 3-methoxy-4-heptanal, 3-nitrobutyl, 4-isopropoxydodecyl, 4-azido-2-nitrodecyl, 5-mercaptononyl, 4-amino-1-pentenyl as well as other substituted groups. Representative alkyl substituents are disclosed in U.S. Pat. No. 5,212,295, at column 12, lines 41-50, hereby incorporated by reference in its entirety.

A number of chemical functional groups can be introduced into compounds of the invention in a blocked form and subsequently deblocked to form a final, desired compound. Such groups can be directly or indirectly attached at the heterocyclic bases, the internucleoside linkages and the sugar substituent groups at one or more of the 2′, 3′ and 5′-positions. Protecting groups can be selected to block functional groups located in a growing oligomeric compound during iterative oligonucleotide synthesis while other positions can be selectively deblocked as needed. In general, a blocking group renders a chemical functionality of a larger molecule inert to specific reaction conditions and can later be removed from such functionality without substantially damaging the remainder of the molecule (Greene and Wuts, Protective Groups in Organic Synthesis, 3rd ed, John Wiley & Sons, New York, 1999). For example, the nitrogen atom of amino groups can be blocked as phthalimido groups, as 9-fluorenylmethoxycarbonyl (FMOC) groups, and with triphenylmethylsulfenyl, t-BOC or benzyl groups. Carboxyl groups can be blocked as acetyl groups. Representative hydroxyl protecting groups are described by Beaucage et al., Tetrahedron 1992, 48, 2223. Preferred hydroxyl protecting groups are acid-labile, such as the trityl, monomethoxytrityl, dimethoxytrityl, trimnethoxytrityl, 9-phenylxanthine-9-yl (Pixyl) and 9-(p-methoxyphenyl)xanthine-9-yl (MOX).

Chemical functional groups can also be “blocked” by including them in a precursor form. Thus, an azido group can be considered a “blocked” form of an amine since the azido group is easily converted to the amine. Further representative protecting groups utilized in oligonucleotide synthesis are discussed in Agrawal, et al., Protocols for Oligonucleotide Conjugates, Eds, Humana Press; New Jersey, 1994; Vol. 26 pp. 1-72.

Examples of hydroxyl protecting groups include, but are not limited to, t-butyl, t-butoxymethyl, methoxymethyl, tetrahydropyranyl, 1-ethoxyethyl, 1-(2-chloroethyoxy)ethyl, 2-trimethylsilylethyl, p-chlorophenyl, 2,4-dinitrophenyl, benzyl, 2,6-dichlorobenzyl, diphenylmethyl, p,p=-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

Examples of thiol (sulfur) protecting groups include, but are not limited to, benzyl, substituted benzyls, diphenylmethly, phenyl, t-butyl, methoxymethyl, thiazolidines, acetyl and benzoyl. Further thiol protecting groups are illustrated in Greene and Wuts, ibid.

Additional amino-protecting groups include but are not limited to, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide-protecting groups, such as formyl, acetyl, trihaloacetyl, benzoyl, and nitrophenylacetyl; sulfonamide-protecting groups, such as 2-nitrobenzenesulfonyl; and imine- and cyclic imide-protecting groups, such as phthalimido and dithiasuccinoyl. Equivalents of these amino-protecting groups are also encompassed by the compounds and methods of the present invention.

Some preferred amino-protecting groups are stable to acid treatment and can be selectively removed with base treatment, which makes reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p.1), and various substituted sulfonylethyl carbamates exemplified by the Nsc group (Samukov et al., Tetrahedron Lett, 1994, 35:7821; Verhart and Tesser, Rec. Trav. Chim. Pays-Bas, 1987, 107:621).

In some especially preferred embodiments, the nucleoside components of the oligomeric compounds are connected to each other by optionally protected phosphorothioate internucleoside linkages. Representative protecting groups for phosphorus containing internucleoside linkages such as phosphite, phosphodiester and phosphorothioate linages include β-cyanoethyl, diphenylsilylethyl, δ-cyanobutenyl, cyano p-xylyl (CPX), N-methyl-N-trifluoroacetyl ethyl (META), acetoxy phenoxy ethyl (APE) and butene-4-yl groups. See for example U.S. Pat. No. 4,725,677 and Re. 34,069 (β-cyanoethyl); Beaucage, S. L. and Iyer, R. P., Tetrahedron, 49 No. 10, pp. 1925-1963 (1993); Beaucage, S. L. and Iyer, R. P., Tetrahedron, 49 No. 46, pp. 10441-10488 (1993); Beaucage, S. L. and Iyer, R. P., Tetrahedron, 48 No. 12, pp.

The oligomeric compound conjugates in accordance with the invention can be used in diagnostics, therapeutics and as research reagents and kits. The compounds can be used in pharmaceutical compositions by including a suitable pharmaceutically acceptable diluent or carrier. They can further be used for treating organisms having a disease characterized by the undesired production of a protein. The organism should be contacted with an oligomeric compound conjugate having an oligonucleotide sequence that is capable of specifically hybridizing with a strand of nucleic acid encoding the undesirable protein. Treatments of this type can be practiced on a variety of organisms ranging from unicellular prokaryotic and eukaryotic organisms to multicellular eukaryotic organisms. Any organism that utilizes DNA-RNA transcription or RNA-protein translation as a fundamental part of its hereditary, metabolic or cellular control is susceptible to therapeutic and/or prophylactic treatment in accordance with the invention. Seemingly diverse organisms such as bacteria, yeast, protozoa, algae, plants and higher animal forms, including warm-blooded animals, can be treated. Further, each cell of multicellular eukaryotes can be treated, as such cells carry out both DNA-RNA transcription and RNA-protein translation as integral parts of their activity. Furthermore, many of the organelles (e.g., mitochondria and chloroplasts) of eukaryotic cells also include transcription and translation mechanisms. Thus, single cells, cellular populations, or organelles can also be included within the definition of organisms that can be treated with therapeutic or diagnostic oligonucleotides.

For therapeutics, an animal, preferably a human, suspected of having a disease or disorder which can be treated by modulating the expression of a particular target gene is treated by administering oligomeric compound conjugates in accordance with this invention. The oligomeric compound conjugates of the invention can be utilized in pharmaceutical compositions by adding an effective amount of the oligomeric compound conjugates to a suitable pharmaceutically acceptable diluent or carrier. Use of the oligomeric compound conjugates and methods of the invention may also be useful prophylactically, e.g., to prevent or delay infection, inflammation or tumor formation, for example.

The oligomeric compound conjugates of the invention are useful for research and diagnostics, because these compounds can be prepared to hybridize to nucleic acids encoding a particular protein, enabling sandwich and other assays to easily be constructed to exploit this fact. Hybridization of the oligomeric compound conjugates of the invention with a nucleic acid encoding a particular protein can be detected by means known in the art. Such means may include conjugation of an enzyme to an oligomeric compound conjugate, radiolabelling of the oligomeric compound conjugate, or any other suitable detection means. Kits using such detection means for detecting protein levels in a sample may also be prepared.

The methods of the invention can be used in connection with diagnostics and therapeutics. Methods in accordance with the invention can be used to improve the permeation of biological membranes by therapeutic and diagnostic oligomeric compounds. Further, the methods of the invention can be used to improve the cellular distribution of therapeutic and diagnostic oligomeric compound conjugates once the compounds penetrate biological membranes.

The present invention also includes pharmaceutical compositions and formulations that include the oligomeric compound conjugates of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligomeric compound conjugates of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and diolcoylphosphatidyl ethanolamine DOTMA). Oligomeric compound conjugates of the invention may be encapsulated within liposomes or may form complexes thereto, in particular to cationic liposomes. Alternatively, oligomeric compound conjugates may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligomeric compound conjugates of the invention are administered in conjunction with one or more penetration enhancers, surfactants, and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium).

Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligomenrc compound conjugates of the invention may be delivered orally in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyomithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. application Ser. Nos. 08/886,829 (filed Jul. 1, 1997), 09/108,673 (filed Jul. 1, 1998), 09/256,515 (filed Feb. 23, 1999), 09/082,624 (filed May 21, 1998) and 09/315,298 (filed May 20, 1999) each of which is incorporated herein by reference in its entirety.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention can be formulated into any of many possible dosage forms such as, but not limited to, tablets, capsules, gel capsules, liquid syrups, soft gels, suppositories, and enemas. The compositions of the present invention can also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension can also contain stabilizers.

In one embodiment of the present invention, the pharmaceutical compositions are formulated and used as foams. Pharmaceutical foams include formulations such as, but not limited to, emulsions, microemulsions, creams, jellies and liposomes. While basically similar in nature, these formulations vary in the components and the consistency of the final product. The preparation of such compositions and formulations is generally known to those skilled in the pharmaceutical and formulation arts and may be applied to the formulation of the compositions of the present invention.

The compositions of the present invention can be prepared and formulated as emulsions. Emulsions are typically heterogenous systems of one liquid dispersed in another in the form of droplets usually exceeding 0.1 μm in diameter. (Idson, in Pharmaceutical Dosage Forms, Liebermnan, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions can contain components in addition to the dispersed phases, and the active drug can be present as a solution in either the aqueous phase, oily phase or as a separate phase. Pharmaceutical excipients such as emulsifiers, stabilizers, dyes, and anti-oxidants can also be present in emulsions. Pharmaceutical emulsions can also comprise more than two phases, such as, for example oil-in-water-in-oil (o/w/o) and water-in-oil-in-water (w/o/w) emulsions. Such complex formulations often provide advantages that are not achieved with simple binary emulsions. Multiple emulsions in which individual oil droplets of an o/w emulsion enclose small water droplets constitute a w/o/w emulsion. Likewise, a system of oil droplets enclosed in globules of water stabilized in an oily continuous provides an o/w/o emulsion.

Emulsions are characterized by little or no thermodynamic stability. Often, the dispersed or discontinuous phase of the emulsion is dispersed into the external or continuous phase and maintained in this form through the action of emulsifiers or the viscosity of the formulation. Either phase of the emulsion can be a semisolid or a solid, as is the case with emulsion-style ointment bases and creams. Other means of stabilizing emulsions entail the use of emulsifiers that can be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants for the preparation of formulations. Surfactants may be classified based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases, such as anhydrous lanolin and hydrophilic petrolatum, can soak up water to form w/o emulsions, yet retain their semisolid consistencies. Finely divided solids have also been used as emulsifiers, especially in combination with surfactants and in viscous preparations. Such solids include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. Such materials include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). Hydrocolloids disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, emulsion formulations often incorporate preservatives. Preservatives commonly added to emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants can be free radical scavengers such as tocopherols, alkyl gallates, butylated hydroxyanisole, butylated hydroxytoluene, or reducing agents, such as ascorbic acid and sodium metabisulfite, and antioxidant synergists such as citric acid, tartaric acid, and lecithin.

The application of emulsion formulations via dermatological, oral and parenteral routes, and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation and efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligomeric compound conjugates are formulated as microemulsions. A microemulsion may be defined as a system of water, oil and amphiphile that is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), and decaglycerol decaoleate (DAO750), alone or in combination with cosurfactants. The cosurfactant, usually a short-chain alcohol such as ethanol, 1-propanol, or 1-butanol, serves to increase the interfacial fluidity by penetrating the surfactant film and creating a disordered film that results from the void space generated among surfactant molecules. Microemulsions can, however, be prepared without the use of cosurfactants, and alcohol-free self-emulsifying microemulsion systems are known in the art. The aqueous phase can include, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are of particular interest from the standpoint of drug solubilization and the enhanced absorption of drugs. It has been proposed that lipid based microemulsions (both o/w and w/o) enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Microemulsions often form spontaneously when their components are brought together at ambient temperature, which may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligomeric compound conjugates from the gastrointestinal tract, as well as improve the local cellular uptake of oligomeric compound conjugates within the gastrointestinal tract, vagina, buccal cavity and other areas of administration.

Microemulsions of the present invention may also contain additional components and additives such as sorbitan monostearate (Grill 3), Labrasol, and penetration enhancers that improve the properties of the formulation and enhance the absorption of the oligomeric compound conjugates of the present invention. Penetration enhancers used in the microemulsions of the present invention may be classified as belonging to one of five broad categories-surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Many organized surfactant structures other than microemulsions exist and have been studied and used for the formulation of drugs. Such structures include monolayers, micelles, bilayers and vesicles. Vesicles, such as liposomes, have attracted great interest due to their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles that have a membrane formed from a lipophilic material and an aqueous interior. The aqueous portion contains the composition to be delivered. Cationic liposomes can fuse to the cell wall. Non-cationic liposomes, although not able to fuse as efficiently with the cell wall, are taken up by macrophages in vivo. In order to cross intact mammalian skin, lipid vesicles must pass through a series of fine pores, each with a diameter less than 50 nm, under the influence of a suitable transdermal gradient. Therefore, it is desirable to use a liposome that is highly deformable and able to pass through such fine pores.

Further advantages of liposomes include biocompatability and biodegradability, the ability to incorporate a wide range of water and lipid soluble drugs, and the ability to protect encapsulated drugs from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size, and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as a mode of delivery for many drugs. Growing evidence indicates that liposomes present several advantages relative to other formulations for topical administration. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes that interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes that are pH-sensitive or negatively-charged entrap DNA, rather than forming a complex with DNA. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765). Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. Nos. 5,540,935 (Miyazaki et al.) and 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates that are attractive candidates for drug delivery vehicles. Transfersomes may be described as lipid droplets that are so highly deformable that they are easily able to penetrate through pores that are smaller than the droplet. Transfersomes are adaptable to the environment in which they are used, e.g. they are self-optimizing (adaptive to the shape of pores in the skin), self-repairing, frequently reach their targets without fragmenting, and are often self-loading. To make transfersomes, surface edge-activators, usually surfactants, are added to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide pH range. In general the HLB values of non-ionic surfactants range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides. The use of surfactants in drug products, formulations and in emulsions has been reviewed (Rieger, in Pharmaceutical Dosage Forms, Marcel Dekker, Inc., New York, N.Y., 1988, p. 285).

In one embodiment, the present invention employs various penetration enhancers to effect the efficient delivery of nucleic acids, particularly oligomeric compound conjugates, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p.92). Each of the above mentioned classes of penetration enhancers are described below in greater detail.

In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligomeric compound conjugates through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Various fatty acids and their derivatives that act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcamitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present invention, can be defined as compounds that remove metallic ions from solution by forming complexes therewith, with the result that absorption of oligonucleotides through the mucosa is enhanced. With regard to the use of chelating agents as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating, non-surfactant penetration enhancing compounds can be defined as compounds that demonstrate insignificant activity as chelating agents or as surfactants, but that nonetheless enhance absorption of oligomeric compound conjugates through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligomeric compound conjugates at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligomeric compounds.

Other agents may be utilized to enhance the penetration of the administered oligomeric compound conjugates, including glycols such as ethylene glycol and propylene glycol, pyrrols such as 2-pyrrol, azones, and terpenes such as limonene and menthone.

Certain compositions of the present invention also incorporate carrier compounds in the formulation. As used herein, “carrier compound” or “carrier” can refer to a nucleic acid, or analog thereof, that is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of an oligomeric compound conjugate and a carrier compound, typically with an excess of the latter substance, can result in a substantial reduction of the amount of oligomeric compound conjugate recovered in the liver, kidney or other extracirculatory reservoirs, presumably due to competition between the carrier compound and the oligomeric compound conjugate for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

In contrast to a carrier compound, a “pharmaceutical carrier” or “excipient” is a pharmaceutically acceptable solvent, suspending agent or any other pharmacologically inert vehicle for delivering one or more oligomeric compound conjugates to an animal. The excipient may be liquid or solid and is selected, with the planned manner of administration in mind, so as to provide for the desired bulk, consistency, etc., when combined with an oligomeric compound conjugate and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with oligomeric compound conjugates can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of oligomeric compound conjugates may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration that do not deleteriously react with oligomeric compound conjugates can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligomeric compound conjugates of the formulation.

Aqueous suspensions may contain substances that increase the viscosity of the suspension including, for example, sodium carboxymethylcellulose, sorbitol and/or dextran. The suspension may also contain stabilizers.

The compounds of the invention may also be admixed, encapsulated, conjugated or otherwise associated with other molecules, molecule structures or mixtures of compounds, such as, for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Certain embodiments of the invention provide pharmaceutical compositions containing (a) one or more oligomeric compound conjugates and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabinc, teniposide, cisplatin and diethylstilbestrol (DES). See, generally, The Merck Manual of Diagnosis and Therapy, 15th Ed. 1987, pp. 1206-1228, Berkow et al., eds., Rahway, N.J. When used with the oligomeric compound conjugates of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more oligomeric compound conjugates targeted to a first nucleic acid and one or more additional oligomeric compound conjugates targeted to a second nucleic acid target. The two or more combined oligomeric compound conjugates may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

The oligomeric compound conjugates of the invention encompass pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes pharmaceutically acceptable salt of an acid form of one of the components of the compositions of the invention. Such salts include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids. Such inorganic acids include, for example, hydrobromic acid, sulfuric acid or phosphoric acid. Such organic acids include, for example, carboxylic, sulfonic, sulfo or phospho acids or N-substituted sulfamic acids, such as, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and amino acids, such as the 20 alpha-amino acids involved in the synthesis of proteins in nature, such as, for example, glutamic acid or aspartic acid, and also phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

Preferred examples of pharmaceutically acceptable salts for oligomeric compound conjugates include, but are not limited to, (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The materials, methods, and examples presented herein are intended to be illustrative, and are not intended to limit the scope of the invention. All publications, patent applications, patents, and other references mentioned herein are incorporated by reference in their entirety. Unless otherwise defined, all technical and scientific terms are intended to have their art-recognized meanings.

### EXAMPLES

### Example 1

Synthesis of Derivatized Solid Support

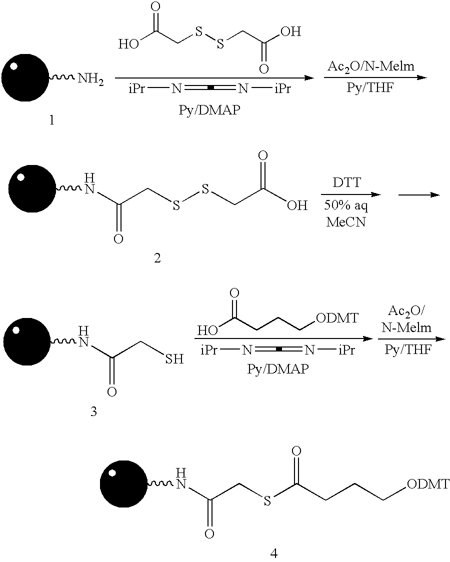

Long chain alkylamine controlled pore glass 1 was first treated with 2,2′-dithiodiglycolic acid, N,N-diisopropylcarbodiimide (5 equiv. each), and 4-dimethylaminopyridine (DMAP, 0.1 equiv.) in pyridine for 2 h. The product, 2, was capped with Ac₂O/N-methylimidazole/Py/THF, and the disulfide bond was reduced with 0.1 M 1,4-dithiothreitol (DTT) in 50% aqueous MeCN for 4 h to give 3. The solid support 3 was treated with 4-(4,4′-dimethoxytrityloxy)butyric acid essentially as described above for the acylation of 1 and finally capped with Ac₂O/N-methylimidazole/Py/THF to yield the solid support 4 loaded at 101-102 μmol g⁻¹ as evidenced by the dimethoxytrityl assay.

### Example 2

Synthesis of Oligonucleotides on Convertible Solid Support 1.

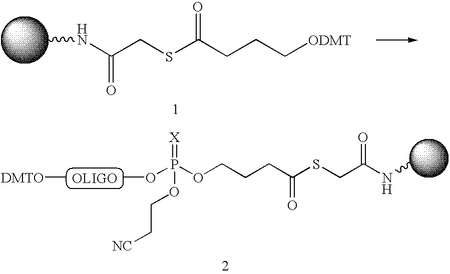

The oligonucleotide synthesis was performed on an ABI 380B DNA Synthesizer on a 1 to 30 μmol scale according to the manufacturer's recommendations. The standard and 2′-O-(2-methoxyethyl) phosphoramidites were used as 0.1 M solutions in anhydrous MeCN. The oxidation step was carried out with the standard iodine reagent or with t-butyl hydroperoxide (10% in MeCN) for 10 min. The preparation of oligonucleotide phosphorothioates was carried out using 3H-1,2-benzodithiol-3-one 1,1-dioxide (0.05 M in MeCN) as a sulfur-transfer reagent. The coupling time of 10 min was used for 2′-O-(2-methoxyethyl) phosphoramidites.

### Example 3

Conjugation of Polyethylenimines to Support-Bound Oligonucleotides 2.

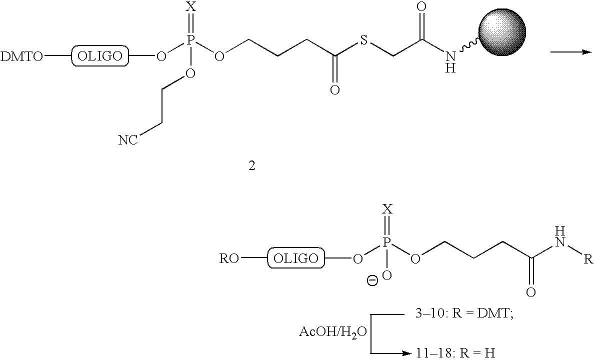

A solid support-bound, protected oligonucleotide 2, (1 to 30 μmol) was shaken with a solution of 25% polyethylenimine and 1 M thiophenol in 30% aqueous MeCN (0.2 mL per μmol) for 1 h at room temperature. The reaction mixture was diluted with concentrated aqueous ammonium hydroxide (1 mL per μmol) and kept at 60° C. for 12 h. The solid phase was removed by filtration or centrifugation, the solution was evaporated. The residue was re-dissolved in water (1 mL per μmol) and filtered again to give solutions of crude 3-10 (Table 1).

TABLE 1

Oligonucleotide conjugates with spermine and polyethylenimines.

| | | | | |
|---|---|---|---|---|
| Compound | Sequence | X | R | R₁ |

| | | | | | |
|---|---|---|---|---|---|
| 3 | T₂₀ | (SEQ ID NO:1) | O | DMT | spermine |
| 4 | T₂₀ | (SEQ ID NO:2) | O | DMT | tetraethylenpentamine |
| 5 | (TG)₂ CTAT₂C(TG)₂ A₂T₂ | (SEQ ID NO:3) | S | DMT | tetraethylenpentamine |
| 6 | T₂₀ | (SEQ ID NO:4) | O | DMT | PEI 600 |
| 7 | (TG)₂ CTAT₂C(TG)₂ A₂T₂ | (SEQ ID NO:5) | S | DMT | PEI 600 |
| 8 | T₂₀ | (SEQ ID NO:6) | O | DMT | PEI 1200 |
| 9 | (TG)₂ CTAT₂C(TG)₂ A₂T₂ | (SEQ ID NO:7) | S | DMT | PEI 1200 |
| 10 | (TG)₂ CTAT₂C(TG)₂ A₂T₂ | (SEQ ID NO:8) | S | DMT | PEI 2000 |
| 11 | T₂₀ | (SEQ ID NO:1) | O | H | spermine |
| 12 | T₂₀ | (SEQ ID NO:2) | O | H | tetraethylenpentamine |
| 13 | (TG)₂ CTAT₂C(TG)₂ A₂T₂ | (SEQ ID NO:3) | S | H | tetraethylenpentamine |
| 14 | T₂₀ | (SEQ ID NO:4) | O | H | PEI 600 |
| 15 | (TG)₂ CTAT₂C(TG)₂ A₂T₂ | (SEQ ID NO:5) | S | H | PEI 600 |
| 16 | T₂₀ | (SEQ ID NO:6) | O | H | PEI 1200 |
| 17 | (TG)₂ CTAT₂C(TG)₂ A₂T₂ | (SEQ ID NO:7) | S | H | PEI 1200 |
| 18 | (TG)₂ CTAT₂C(TG)₂ A₂T₂ | (SEQ ID NO:8) | S | H | PEI 2000 |

### Example 4

Purification of Oligonucleotides Conjugates 3-10.

The solutions from previous step containing 3-10 were cooled in an ice bath and neutralized with 50% aqueous AcOH. The precipitate of crude oligonucleotide conjugates complexed with excess polyethylenimine was collected by centrifugation, washed with MeCN (5×20 mL) and ether (5×20 mL), and re-dissolved in a mixture of piperidine and DMSO (9:1; 20 mL). The oligonucleotides were first purified on a Sephadex G25 column (500 mL) eluting with 30% aqueous MeCN.

Conjugates 3-5 were readily isolated on a C4 column under the standard condition of the DMT-On purification. Isolation of conjugates 6-10 required adding a strong chaotropic agent, NH4Br, to the standard HPLC buffer. Thus, a Zorbax C3 column was eluted at 55° C. with 0.1 M NH4OAc as buffer A and 1.5 M NH4Br+0.1 M NH4OAc in 40% aqueous MeCN as buffer B using a linear gradient from 0 to 100% B for 45 min. Under these conditions, the crude 6-10 were readily purified from DMT-Off truncated oligonucleotides.

The purified 3-10 were acidified with AcOH to pH 4.2 and kept for 2 days, which effected the complete removal of the 5′-teminal DMT group. Upon neutralization with concentrated aqueous ammonium hydroxide to pH 8.0, the solutions of oligonucleotides 11-18 were concentrated in vacuo, extracted with ethyl acetate (2×50 mL), and dialyzed in Spectra/Pore® 2000 cellulose dialysis bags (cutoff limit 2000 Da) against water (3×1 L) and 0.1 M NaOAc (pH 7.8; 1 L). The dialyzed solutions were finally desalted on a Sephadex G25 column eluting with 30% aqueous MeCN to give pure oligonucleotides 11-18, which were readily soluble in water and were characterized by ES MS. The conjugates 14-18 showed the expected sets of peaks with an increment of 43.1 au.

### Example 5

Reactive Solid Support 20.

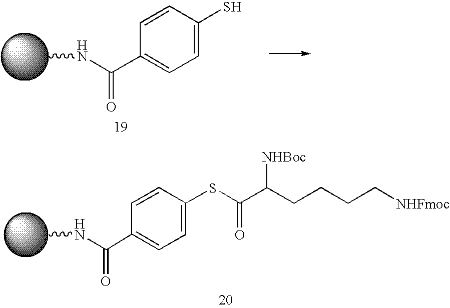

A mixture of α-BOC-ε-FMOC-L-Lysine (0.3 mmol), thiophenyl-derivatized polystyrene 19 (100 μmol), and pyridine (25 mL) is treated with N,N′-diisopropyl carbodiimide (0.4 mmol) for 12 h at room temperature. The liquid phase is filtered off, and the solid support is capped by treating with a mixture of Ac₂O/pyridine/N-methylimidazole/THF (10:10:10:70) for 3 h at room temperature. Finally, the solid support 20 is washed with MeCN and ethyl acetate and dried.

### Example 6

Reactive Solid Support 21.

A mixture of α-BOC-ε-FMOC-L-Lysine (0.3 mmol), thiophenyl-derivatized PS—PEG (100 μmol), and pyridine (25 mL) is treated with N,N′-diisopropyl carbodiimide (0.4 mmol) for 12 h at room temperature. The liquid phase is filtered off, and the solid support is capped by treating with a mixture of Ac₂O/pyridine/N-methylimidazole/THF (10:10:10:70) for 3 h at room temperature. Finally, the solid support 21 is washed with MeCN and ethyl acetate and dried.

### Example 7

Reactive Solid Support 22.

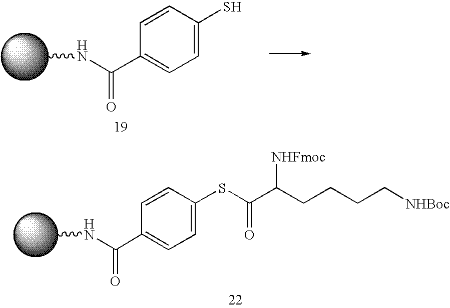

A mixture of α-FMOC-ε-BOC-L-Lysine (0.3 mmol), thiophenyl-derivatized polystyrene 19 (100 μmol), and pyridine (25 mL) is treated with N,N′-diisopropyl carbodiimide (0.4 mmol) for 12 h at room temperature. The liquid phase is filtered off, and the solid support is capped by treating with a mixture of Ac₂O/pyridine/N-methylimidazole/THF (10:10:10:70) for 3 h at room temperature. Finally, the solid support 22 is washed with MeCN and ethyl acetate and dried.

### Example 8

Reactive Solid Support 23.

A mixture of N—FMOC-glycine (0.3 mmol), thiophenyl-derivatized polystyrene (100 μmol), and pyridine (25 mL) is treated with N,N′-diisopropyl carbodiimide (0.4 mmol) for 12 h at room temperature. The liquid phase is filtered off, and the solid support is capped by treating with a mixture of Ac₂O/pyridine/N-methylimidazole/THF (10:10:10:70) for 3 h at room temperature. Finally, the solid support 23 is washed with MeCN and ethyl acetate and dried.

### Example 9

Conjugation of Polyethylenimines to Support-Bound PNA 2.

The PNA synthesis is performed on an ABI 380B DNA Synthesizer on a 1 to 30 μmol scale according to the manufacturer's recommendations using solid supports 20-23. The support-bound material is deprotected by treatment with trifluoroacetic acid (TFA) and trimethylsilane and washed with methylene dichloride and MeCN. The solid phase is then treated with 25% aqueous polyethylenimine 600, 1200, or 2000 for 12 h at 50° C. The liquid phase is collected, diluted with water, and brought to pH 2 with 50% aqueous TFA. The title compounds are isolated by reverse-phase HPLC using a gradient of MeCN in 2% aqueous TFA.

### Example 10

N,N′-bis(2,4-dinitrobenzenesulfonyl)-N,N′-bis(2-((4-methoxytrityl)amino)ethyl)-1,2-ethanediamine (25).

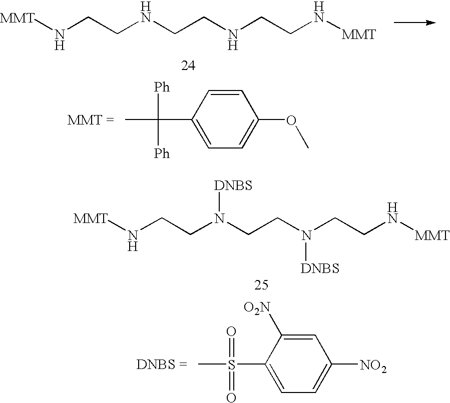

A solution of compound 24 (69.5 g, 0.1 mmol) in pyridine (300 mL) and diisopropylethylamine (40 mL) was treated with 2,4-dinitrobenzenesulfonyl chloride (58.7 g, 0.22 mol) overnight at room temperature. The solvent was evaporated. The residue was dissolved in ethyl acetate and washed with 5% aqueous NaHCO₃ and brine (3×100 mL) and dried over Na₂SO₄. The product was purified on a silica gel column eluting with a step gradient of methanol in CH₂Cl₂ (0 to 20%). The title compound 25 (100.5 g, 87%) was obtained as a colorless powder.

### Example 11

3,6,9,12-tetra-(2,4-dinitrobenzenesulfonyl)-3,6,9,12-tetraaza-1,14-bis((4-methoxytrityl)amino)tetradecane (28).

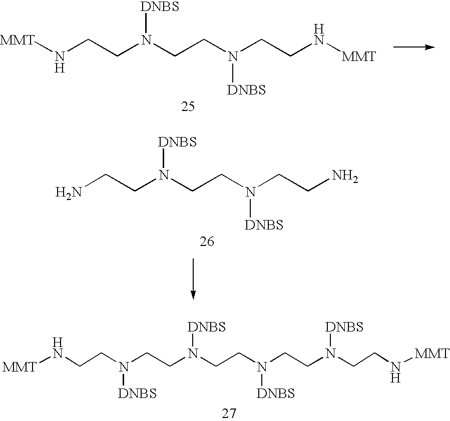

The compound 25 (11.5 g, 10 mmol) was treated with dichloroacetic acid (2% in CH₂Cl₂/MeOH; 9:1; 100 mL) for 20 min at room temperature. The solution was washed with 5% aqueous NaHCO₃ and brine (3×100 mL) and dried over Na₂SO₄. On evaporation, the residue was dissolved in ethyl acetate (50 mL) and precipitated into diethyl ether (300 mL). The precipitate of 3,6-bis-(2,4-dinitrobenzenesulfonyl)-3,6-diaza-1,8-diaminooctane 26 was dried and used without further purification.

A solution of this in pyridine was treated with 2-((4-methoxytrityl)amino)acetic aldehyde (g, mL) for 2 h at room temperature followed by treatment with sodium cyanoborohydride (mg, mmol) and 4-toluenesulfonic acid (mg, mmol) overnight at room temperature. The solvent was evaporated, the residue was dissolved in ethyl acetate washed with 5% aqueous NaHCO₃ and brine (3×100 mL) and dried over Na₂SO₄. The product was purified on a silica gel column eluting with a step gradient of methanol in CH₂Cl₂ (0 to 20%). The title compound 27 (11.4 g, 61%) was obtained as a colorless powder.

### Example 12

Bis-triethylammonium 3,6,9,12,15,18-hexa(2,4-dinitrobenzenesulfonyl)-3,6,9,12,15,18-hexaaza-1,20-eicosadicarboxylate (28).

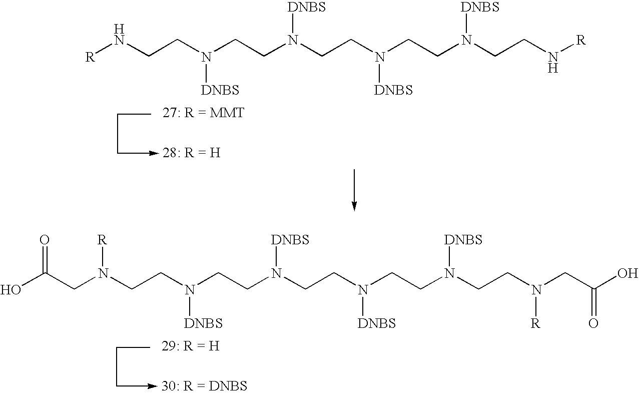

The compound 27 (17.0 g, 10 mmol) is treated with dichloroacetic acid (2% in CH₂Cl₂/MeOH; 9:1; 100 mL) for 20 min at room temperature. The solution is washed with 5% aqueous NaHCO₃ and brine (3×100 mL) and dried over Na₂SO₄. On evaporation, the residue is dissolved in ethyl acetate (50 mL) and precipitated into diethyl ether (300 mL). The precipitate of 3,6,9,12-tetra-(2,4-dinitrobenzenesulfonyl)-3,6,9,12-tetraaza-1,14-diaminotetradecane 28 is dried and used without further purification.

A solution of this in CH₂Cl₂ (80 mL) is treated with 2-bromoacetic acid (3.1 g, 22 mmol) and ethyldiisopropylamine (5 mL) overnight at room temperature and evaporated, the residue is dissolved in ethyl acetate, washed with 2% aqueous HCl (3×50 mL) and water and dried over Na₂SO₄. Upon evaporation, the crude compound 29 is dissolved in pyridine (100 mL) and diisopropylethylamine (5 mL) and treated with 2,4-dinitrobenzenesulfonyl chloride (5.9 g, 22 mmol) overnight at room temperature. The solvent is evaporated. The residue is dissolved in ethyl acetate and washed with 1 M aqueous triethylammonium acetata (3×50 mL) and dried over Na₂SO₄. The product is purified on a silica gel column eluting with a step gradient of methanol (0 to 30%) in CH₂Cl₂ plus 5% triethylamine. The title compound 30 is obtained as a triethylammonium salt.

### Example 13

3,6,9,12,15,18,21,24,27,30,33,36-Dodecaaza-10,29-dioxo-1,38-diaminooctatriacontane (32).

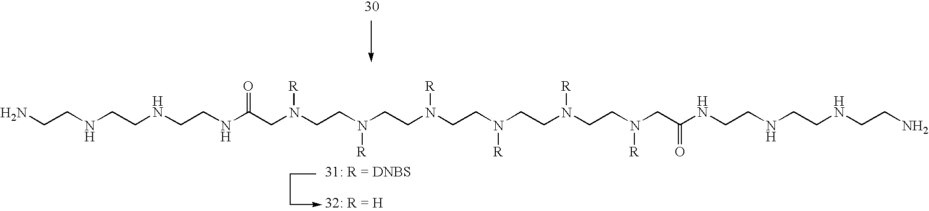

A solution of 30 (3.86 g, 2.0 mmol) in pyridine (25 mL) is treated first with HATU (0.80 g, 2.1 mmol) for 20 min followed by addition of triethylenetetramine (0.58 g, 4.0 mmol) and stirring for 2 h at room temperature. The solvent is evaporated, crude 31 is treated with concentrated aqueous ammonium hydroxide overnight at room temperature. The mixture is evaporated, dissolved in 1% aqueous HCl, and extracted with ethyl acetate (3×50 mL). The aqueous layer is applied on a Dowex CCR-3 (H⁺) column, and the product is eluted with a linear gradient of ammonium hydroxide (0 to 25%). The fractions are evaporated to give 32 as an oil.

### EXAMPLE 14

Conjugation of Solid Support-Bound Oligonucleotides to 32.

A solid support-bound, protected oligonucleotide 2, (1 to 30 μmol) is shaken with a solution of 25% 32 and 1 M thiophenol in 30% aqueous MeCN (0.2 mL per μmol) for 1 h at room temperature. The reaction mixture is diluted with concentrated aqueous ammonium hydroxide (1 mL per μmol) and kept at 60° C. for 12 h. The solid phase is removed by filtration or centrifugation, the solution is evaporated. The residue is re-dissolved in water (1 mL per μmol) and filtered again to give a solution of crude product.

The solutions is cooled in an ice bath and neutralized with 50% aqueous AcOH. The precipitate of crude oligonucleotide conjugate complexed with excess 32 is collected by centrifugation, washed with MeCN (5×20 mL) and ether (5×20 mL), and re-dissolved in a mixture of piperidine and DMSO (9:1; 20 mL). The oligonucleotides were first purified on a Sephadex G25 column (500 mL) eluting with 30% aqueous MeCN. The conjugate is isolated on a Zorbax C3 column eluted at 55° C. with 0.1 M NH4OAc as buffer A and 1.5 M NH4Br+0.1 M NH4OAc in 40% aqueous MeCN as buffer B using a linear gradient from 0 to 100% B for 45 min.

The purified conjugate is acidified with AcOH to pH 4.2 and kept for 2 days. Upon neutralization with concentrated aqueous ammonium hydroxide to pH 8.0, the solution is concentrated in vacuo, extracted with ethyl acetate (2×50 mL), and dialyzed in a Spectra/Pore® 2000 cellulose dialysis bag (cutoff limit 2000 Da) against water (3×1 L) and 0.1 M NaOAc (pH 7.8; 1 L). The dialyzed solution is finally desalted on a Sephadex G25 column eluting with 30% aqueous MeCN to give pure conjugate of an oligonucleotide with 32.

### Example 15

Triethylammonium 11-((4-methoxytrityl)amino)-3,6,9-tris(2,4-dinitrophenylsulfonul)-3,6,9-triazaundecanoate (35).

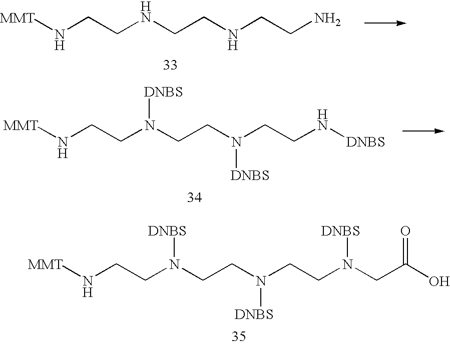

A solution of 33 (4.2.0 g, 10 mol) in pyridine (40 mL) and ethyldiisopropylamine (6 mL) was treated with2,4-dinitrobenzenesulfonyl chloride (9.33 g, 35 mmol) overnight at room temperature. The solvent was evaporated. The residue was dissolved in ethyl acetate, washed with 5% aqueous NaHCO₃ and brine (3×100 mL) and dried over Na₂SO₄. The product was purified on a silica gel column eluting with a step gradient of methanol in CH₂Cl₂ (0 to 20%). The compound 34 (9.11 g, 82%) was obtained as a colorless powder.

A solution of 34 (11.1 g, 10 mmol) in CH₂Cl₂ (80 mL) is treated with 2-bromoacetic acid (1.7 g, 12 mmol) and ethyldiisopropylamine (2.5 mL) overnight at room temperature and evaporated, the residue is dissolved in ethyl acetate, washed with 1 M aqueous triethylammonium acetata (3×50 mL) and dried over Na₂SO₄. The product is purified on a silica gel column eluting with a step gradient of methanol (0 to 30%) in CH₂Cl₂ plus 5% triethylamine. The title compound 35 is obtained as a triethylammonium salt.

### Example 16

Preparation of Oligonucleotide Conjugates with Polyamines of Defined Length by Sequential Synthesis on Solid Phase.

The building block 35 is coupled to the solid support 23 using HATU/HOBT in N-methylpirrolidone as a coupling agent. Upon washing the resin with N-methylpirrolidone and CH₂Cl₂, the terminal MMT group is removed with 2% dichloroacetic acid in CH₂Cl₂, which completes the coupling cycle. After the desired number of the coupling cycles is carried out, 4-((4,4′-dimethoxytrityl)oxy)butyric acid is attached to the terminal amino group in a similar manner.

The synthesis of the oligonucleotide part of the conjugate is then performed using phosphoramidite chemistry and the standard oligonucleotide coupling cycle.

Upon completion of the chain assembly, the solid support is treated with ammonium hydroxide plus 0.25 M dithiothreitol for 8 h at 55° C. The solution is evaporated, and the crude product is isolated on a Zorbax C3 column eluted at 55° C. with 0.1 M NH4OAc as buffer A and 1.5 M NH4Br+0.1 M NH4OAc in 40% aqueous MeCN as buffer B using a linear gradient from 0 to 100% B for 45 min. The purified conjugate is acidified with AcOH to pH 4.2 and kept for 2 days. Upon neutralization with concentrated aqueous ammonium hydroxide to pH 8.0, the solution is concentrated in vacuo, extracted with ethyl acetate (2×50 mL), and dialyzed in a Spectra/Pore® 2000 cellulose dialysis bag (cutoff limit 2000 Da) against water (3×1 L) and 0.1 M NaOAc (pH 7.8; 1 L). The dialyzed solution is finally desalted on a Sephadex G25 column eluting with 30% aqueous MeCN to give pure conjugate of an oligonucleotide with a polyethylenimine of the desired length.

### Example 17

Uptake of Polyethylenimine-Antisense Oligonucleotide Conjugates by Lymphoma Cells

Survivin is an inhibitor of apoptosis protein (IAP), which is expressed at inappropriate levels in many cancers. Reed, J. C., et al., Nature Cell Biology, 1999, 1, E199. Apoptosis protein is required for control of apoptosis, as well as for correct cell division. Li, F., et al., Nature Cell Biology, 1999, 1, 461. Polyethylenimines of varying lengths were directly conjugated to an antisense oligonucleotide targeting the survivin gene.

Compound Description:

Five compounds, which were derivatives of the same 20-mer MOE gapmer oligonucleotide targeting surviving, were evaluated. ISIS 212222 was the negative control compound, and had only the linker at the 5′ end of the oligo. ISIS 212223 had five conjugated ethylenimine groups. ISIS 212224, 212225 and 212226 had PEI of MW 600, 1200 and 2000 conjugated, respectively. Each compound was FITC labeled.

Cell Culture and Uptake of Compounds:

Jurkat cells, a T cell lymphoma line, were maintained in RPMI-1640 medium supplemented with 10% FBS, 1% pen/strep and 1% sodium pyruvate, in a 37° C. 5% CO₂ atmosphere. In order to evaluate uptake, 50,000 cells were incubated with the compounds at the appropriate concentration in a total volume of 1 00ml in serum-containing medium, for 24 hours.

FACS Quantitation of Uptake:

After incubation, the cells were washed twice in 200 ml PBS, then resuspended in 200 ml PBS with 10 ml propidium iodide (50 mg/ml in PBS). Live cells were gated by exclusion of PI, and mean fluorescence, indicating uptake of the compounds was reported for this population.

Uptake by FACS:

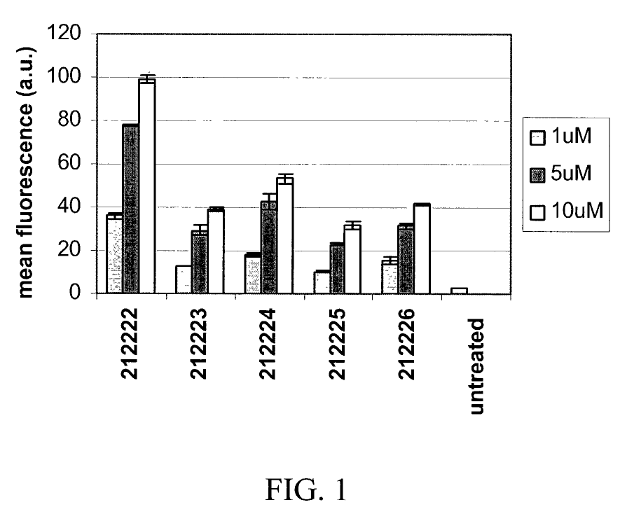

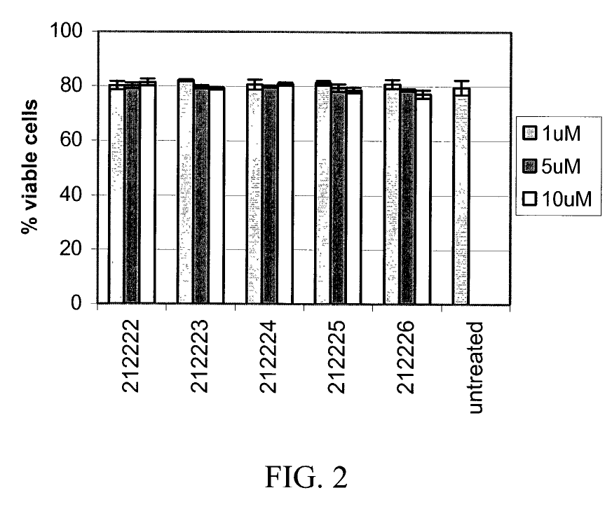

All compounds showed a dose-dependent uptake, with significantly greater fluorescence than untreated cells, as shown in FIG. 1. The control compound, ISIS 212222 was taken up to the largest degree. Cell viability after 24 hours, as determined by the exclusion of propidium iodide, was equivalent to untreated cells for all compounds, at all concentrations, as shown in FIG. 2.

Polyethylenimine conjugated antisense oligonucleotides demonstrated dose-dependent uptake in Jurkat cells, a T cell lymphoma line. These novel compounds did not cause toxicity to the cells after 24 hours of incubation.

### Example 18

4-[(4,4′-dimethoxytrityl)oxy]butyric acid, triethylammonium salt.

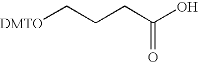

A solution of 4,4′-dimethoxytrityl chloride (6.77 g, 20.0 mmol) and 4-hydroxybutyric acid (252 g, 20.0 mmol) in anhydrous Py (25 mL) was stirred overnight and concentrated to an oil in vacuo. The residue was dissolved in ethyl acetate (200 mL) and washed with water and 2 M aqueous triethylammonium acetate (5×20 mL). The organic solution was evaporated, re-dissolved in CH₂Cl₂, dried over Na₂SO₄, and evaporated to give crude triethylammonium 4-[(4,4′-dimethoxytrityl)oxy]butyrate (9.36 g).

### Example 19

Thioglycolyl-Derivatized CPG.

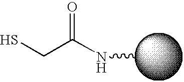

Dithiodiglycolic acid (1.26 g, 6.91 mmol) and N,N′-diisopropylcarbodiimide (1.81 g, 14.0 mmol) were added to a suspension of long chain amino alkyl CPG (6.0 g, 0.691 mmol) in anhydrous Py (40 mL). The suspension was shaken overnight and filtered. The solid support was washed with Py (4×20 mL) and THF (3×20 mL) and treated with a mixture of acetic anhydride (2.0 mL) and N-methylimidazole (4.0 mL) in anhydrous THF (36 mL) for 30 min. Finally, the solid support was extensively washed with THF (5×20 mL) and dried in vacuo.

The solid support from the previous step (6.0 g) was treated with 0.15 M dithiodthreithol in 50% aqueous MeCN (30 mL) for 2 h. The suspension was filtered and briefly washed with 50% aqueous MeCN (5×20 mL) and MeCN (3×20 mL). The thioglycolyl-derivatized CPG obtained was dried in vacuo and used in the following step as soon as possible.

### Example 20

2-[4-[(4,4′-dimethoxytrityl)oxy]butyrylthio]acetyl derivatized CPG

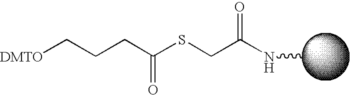

A suspension of solid support (6.0 g) in Py (40 mL) was treated with triethylammonium 4-[(4,4′-dimethoxytrityl)oxy]butyrate (2.94 g, 4.84 mmol) and N,N′-diisopropylcarbodiimide (610 mg, 4.84 mmol). The suspension was shaken overnight and filtered. The solid support was washed with Py (4×20 mL) and THF (3×20 ml.) and treated with a mixture of acetic anhydride (2.0 mL) and N-methylimidazole (4.0 mL) in anhydrous THF (36 mL) for 30 min. Finally, the solid support was extensively washed with THF (5×20 mL) and dried in vacuo. As determined by the dimethoxytrityl assay, the loading of the solid support 5 was 84-87 μmol g⁻¹.

### Example 21

2-[2-(4,4′-Dimethoxytrityloxy)ethoxy]acetic acid, triethylammonium salt.

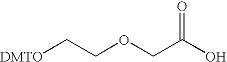

A solution of 2-(2-hydroxyethoxy)acetic acid prepared as desctibed in (Snapp, Thomas C., Jr.; Blood, Alden E. Ether diester derivatives of p-dioxanone. U.S. (1975), 4 pp. U.S. Pat. No. 3,929,847) (7.45 g, 22 mmol) in pyridine (20 mL) is treated with 4,4′-dimethoxytrityl chloride (10.16 g, 30 mmol) overnight at room temperature and concentrated to an oil in vacuo. The residue is dissolved in a mixture of MeOH and CH₂Cl₂ (95:5, v/v; 200 mL) and washed with 2 M aqueous triethylammonium acetate (5×20 mL). The organic solution is evaporated, re-dissolved in CH₂Cl₂, dried over Na₂SO₄, and evaporated to give crude triethylammonium 2-[2-(4,4′-dimethoxytrityloxy)ethoxy] acetate in quantitative yield (10.47 g).

### Example 22

2-[2-[2-(4,4′-Dimethoxytrityloxy)ethoxy]acetylthio]acetyl-Derivatized CPG

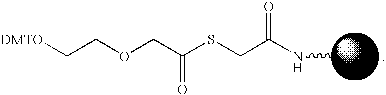

Thioglycolyl-derivatized CPG (2.0 g) prepared as described above is suspended in Py (10 mL) and treated with 4 (1047 mg, 2.0 mmol) and N,N′-diisopropylcarbodiimide (505 mg, 4.0 mmol). The suspension is shaken overnight and filtered. The solid support is washed with Py (4×20 mL) and THF (3×20 mL) and treated with a mixture of acetic anhydride (1.0 mL) and N-methylimidazole (2.0 mL) in anhydrous THF (18 mL) for 30 min. Finally, 2-[2-[2-(4,4′-dimethoxytrityloxy)ethoxy]acetylthio]acetyl-derivatized CPG is extensively washed with THF (5×20 mL) and dried in vacuo.

### Example 23

3-[2-[2-(4,4′-Dimethoxytrityloxy)ethoxy]propionicacid, Triethylammonium Salt.

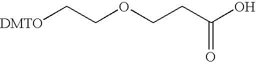

A solution of 2-(2-hydroxyethoxy)acetic acid prepared as desctibed in (Huskens, J.; Peters, J. A.; Van Bekkum, H. The addition of hydroxyl compounds to unsaturated carboxylic acids homogeneously catalyzed by lanthanide(III). Tetrahedron (1993), 49(15), 3149-3164) (1.34 g, 10 mmol) in pyridine (20 mL) is treated with 4,4′-dimethoxytrityl chloride (3.73 g, 11 mmol) overnight at room temperature and concentrated to an oil in vacuo. The residue is dissolved in a mixture of MeOH and CH₂Cl₂ (95:5, v/v; 200 mL) and washed with 2 M aqueous triethylammonium acetate (5×20 mL). The organic solution is evaporated, re-dissolved in CH₂Cl₂, dried over Na₂SO₄, and evaporated to give crude triethylammonium 3-[2-[2-(4,4′-dimethoxytrityloxy) ethoxy]propionate in quantitative yield (5.37 g).

### Example 24

2-[3-[2-[2-(4,4′-Dimethoxytrityloxy)ethoxy]propionylthio]acetyl -Derivatized CPG.

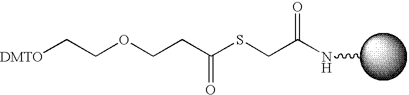

Thioglycolyl-derivatized CPG (2.0 g) prepared as described above is suspended in Py (10 mL) and treated with triethylammonium 3-[2-[2-(4,4′-dimethoxytrityloxy)ethoxy]propionate (1075 mg, 2.0 mmol) and N,N′-diisopropylcarbodiimide (504 mg, 4.0 mmol). The suspension is shaken overnight and filtered. The solid support is washed with Py (4×20 mL) and THF (3×20 mL) and treated with a mixture of acetic anhydride (1.0 mL) and N-methylimidazole (2.0 mL) in anhydrous THF (18 mL) for 30 min. Finally, the product, 2-[3-[2-[2-(4,4′-Dimethoxytrityloxy)ethoxy]propionylthio]acetyl -derivatized CPG, is extensively washed with THF (5×20 mL) and dried in vacuo.

### Example 25

4-mercaptobenzoyl-Derivatized CPG.

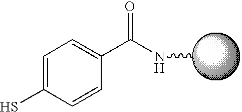

Commercial (4,4′-dithiobis)benzoic acid (674 mg, 2.2 mmol) and N,N′-diisopropylcarbodiimide (555 mg, 4.4 mmol) were added to a suspension of long chain amino alkyl CPG (4.0 g, 0.44 mmol) in anhydrous Py (30 mL). The suspension was shaken overnight and filtered. The solid support was washed with Py (4×50 mL) and THF (3×50 mL) and treated with a mixture of acetic anhydride (2.0 mL) and N-methylimidazole (4.0 mL) in anhydrous THF (36 mL) for 30 min. Finally, the solid support was extensively washed with THF (5×50 mL) and dried in vacuo.

The solid support from the previous step (4 g) was treated with 0.5 M dithiothreithol in a mixture of water, MeCN, and triethylamine (40:20:40; 20 mL) for 2 h. The suspension was filtered and briefly washed with 50% aqueous MeCN (5×30 mL) and THF (3×30 mL). The product, 4-mercaptobenzoyl-derivatized CPG, was dried in vacuo and used in the following step as soon as possible.

### Example 26

4-[4-[(4,4′-dimethoxytrityl)oxy]butyrylthio]benzoyl Derivatized CPG.

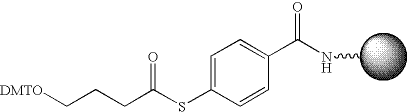

A suspension of 4-mercaptobenzoyl-derivatized CPG (4.0 g) in Py (20 mL) was treated with triethylammonium 4-[(4,4′-dimethoxytrityl)oxy]butyrate (1117 mg, 2.2 mmol) and N,N′-diisopropylcarbodiimide (555 mg, 4.4 mmol). The suspension was shaken overnight and filtered. The solid support was washed with Py (4×40 mL) and THF (3×40 mL) and treated with a mixture of acetic anhydride (2.0 mL) and N-methylimidazole (4.0 mL) in anhydrous THF (36 mL) for 30 min. Finally, the solid support was extensively washed with THF (5×40 mL) and dried in vacuo. As determined by the dimethoxytrityl assay, the loading of 4-[4-[(4,4′-dimethoxytrityl)oxy]butyrylthio]benzoyl derivatized CPG was 70-71 μmol g⁻¹.

### Example 27

3-mercaptobenzoyl-Derivatized CPG.

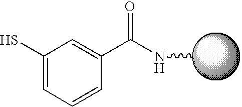

Commercial 3,3′-dithiobis-benzoic acid (674 mg, 2.2 mmol) and N,N′-diisopropylcarbodiimide (555 mg, 4.0 mmol) were added to a suspension of long chain amino alkyl CPG (4.0 g, 0.44 mmol) in anhydrous Py (30 mL). The suspension was shaken overnight and filtered. The solid support was washed with Py (4×40 mL) and THF (3×40 mL) and treated with a mixture of acetic anhydride (2.0 mL) and N-methylimidazole (4.0 mL) in anhydrous THF (36 mL) for 30 min. Finally, the solid support was extensively washed with THF (5×40 mL) and dried in vacuo.

The solid support from the previous step (4 g) was treated with 0.5 M dithiothreithol in a mixture of water, MeCN, and triethylamine (40:20:40; 20 mL) for 2 h. The suspension was filtered and briefly washed with 50% aqueous MeCN (5×40 mL) and THF (3×40 mL). The product, 3-mercaptobenzoyl-derivatized CPG was dried in vacuo and used in the following step as soon as possible.

### Example 28

3-[4-[(4,4′-dimethoxytrityl)oxy]acetylthio]benzoyl Derivatized CPG.

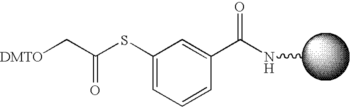

A suspension of 3-mercaptobenzoyl-derivatized CPG (2.0 g, 0.22 mmol) in Py (10 mL) is treated with triethylammonium 4-[(4,4′-dimethoxytrityl)oxy]acetate prepared as described in (Hovinen, J; Guzaev, A.; Azhayev, A.; Lönnberg, H. Tetrahedron, 1994, 50(24), 7203-7218) (528 mg, 1.1 mmol) and N,N′-diisopropylcarbodiimide (278 mg, 2.2 mmol). The suspension is shaken overnight and filtered. The solid support is washed with Py (4×20 mL) and THF (3×20 mL) and treated with a mixture of acetic anhydride (1.0 mL) and N-methylimidazole (2.0 mL) in anhydrous THF (18 mL) for 30 min. Finally, 3-[4-[(4,4′-dimethoxytrityl)oxy]acetylthio]benzoyl derivatized CPG is extensively washed with THF (5×20 mL) and dried in vacuo.

### Example 29

3-[4-[(4,4′-dimethoxytrityl)oxy]butyrylthio]benzoyl Derivatized CPG.

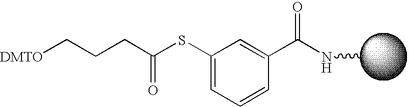

A suspension of 3-mercaptobenzoyl-derivatized CPG (4.0 g) in Py (20 mL) was treated with triethylammonium 4-[(4,4′-dimethoxytrityl)oxy]butyrate (1117 mg, 2.2 mmol) and N,N′-diisopropylcarbodiimide (555 mg, 4.4 mmol). The suspension was shaken overnight and filtered. The solid support was washed with Py (4×40 mL) and THF (3×40 mL) and treated with a mixture of acetic anhydride (2.0 mL) and N-methylimidazole (4.0 mL) in anhydrous THF (36 mL) for 30 min. Finally, the solid support was extensively washed with THF (5×40 mL) and dried in vacuo. As determined by the dimethoxytrityl assay, the loading of the product, 3-[4-[(4,4′-dimethoxytrityl)oxy]butyrylthio]benzoyl derivatized CPG, was 84-86 μmol g⁻¹.

### Example 30

4-[[(4,4′-dimethoxytrityl)oxy]methyl]Benzoic Acid, Triethylammonium Salt.

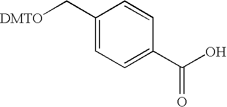

A solution of commercial 4-(hydroxymethyl)benzoic acid (1.52 g, 10.0 mmol) in pyridine (50 mL) is treated with 4,4′-dimethoxytrityl chloride (3.73 g, 11.0 mmol) overnight at room temperature and concentrated to an oil in vacuo. The residue is dissolved in a mixture of MeOH and CH₂Cl₂ (95:5, v/v; 200 mL) and washed with 2 M aqueous triethylammonium acetate (5×20 mL). The organic solution is evaporated, re-dissolved in CH₂Cl₂, dried over Na₂SO₄, and evaporated to give crude triethylammonium 4-[[(4,4′-dimethoxytrityl)oxy]methyl]benzoate in quantitative yield (1016 mg).

### Example 31

2-[4-[[(4,4′-dimethoxytrityl)oxy]methyl]benzoylthio]acetyl-derivatized CPG.

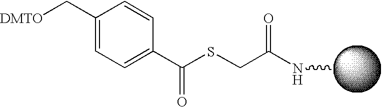

A suspension of thioglycolyl-derivatized CPG prepared as described above (2.0 g) in Py (10 mL) is treated with triethylammonium 4-[[(4,4′-dimethoxytrityl)oxy]methyl]benzoate (611 mg, 1.1 mmol) and N,N′-diisopropylcarbodiimide (278 mg, 2.2 mmol). The suspension is shaken overnight and filtered. The solid support is washed with Py (4×20 mL) and THF (3×20 mL) and treated with a mixture of acetic anhydride (1.0 mL) and N-methylimidazole (2.0 mL) in anhydrous THF (18 mL) for 30 min. Finally, the product, 2-[4-[[(4,4′-dimethoxytrityl)oxy]methyl]benzoylthio]acetyl-derivatized CPG, is extensively washed with THF (5×20 mL) and dried in vacuo.

### Example 32

2-[4-[[(4,4′-dimethoxytrityl)oxy]methyl]benzoylthio]benzoyl-Derivatized CPG

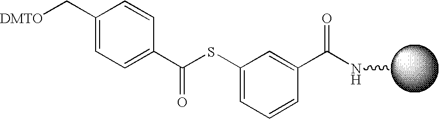

A suspension of 3-mercaptobenzoyl derivatized CPG prepared as described above (2.0 g) in Py (10 mL) is treated with triethylammonium 4-[[(4,4′-dimethoxytrityl)oxy]methyl]benzoate (611 mg, 1.1 mmol) and N,N′-diisopropylcarbodiimide (278 mg, 2.2 mmol). The suspension is shaken overnight and filtered. The solid support is washed with Py (4×20 mL) and THF (3×20 mL) and treated with a mixture of acetic anhydride (1.0 mL) and N-methylimidazole (2.0 mL) in anhydrous THF (18 mL) for 30 min. Finally, the product, 2-[4-[[(4,4′-dimethoxytrityl)oxy]methyl]benzoylthio]benzoyl-derivatized CPG, is extensively washed with THF (5×20 mL) and dried in vacuo.

### Example 33

Conjugate of Polyethylenimine 600 with Oligonucleotide (TG)₂ctat₂c(tg)₂A₂T₂ via 3′-O-[[4-[carbamoyl]phenyl]methyl]thiophosphate linker.

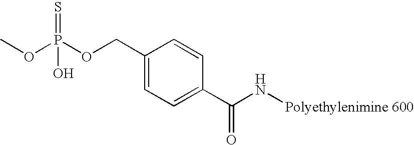

A solid support-bound oligonucleotide TGT₂AT₂CT₃AGA₂TG₂ assembled on 2-[4-[[(4,4′-dimethoxytrityl)oxy]methyl]benzoylthio]acetyl-derivatized CPG (4 μmol) is treated with a solution of 25% polyethylenimine and 1 M thiophenol in 30% aqueous MeCN (0.2 mL per μmol) for 1 h at room temperature. The reaction mixture was diluted with concentrated aqueous ammonium hydroxide (1 mL per μmol) and kept at 60° C. for 12 h. The solid phase was removed by filtration or centrifugation, the solution was evaporated. The residue was re-dissolved in water (1 mL per μmol) and filtered again to give crude 5′-DMT-protected oligonucleotide derivatized with PEI 600. The solution is cooled in an ice bath and neutralized with 50% aqueous AcOH. The precipitate of crude oligonucleotide conjugate complexed with excess polyethylenimine is collected by centrifugation, washed with MeCN (5×20 mL) and ether (5×20 mL), and re-dissolved in a mixture of piperidine and DMSO (9:1; 20 mL). The oligonucleotide is first purified on a Sephadex G25 column (500 mL) eluting with 30% aqueous MeCN. The obtained product is purified on a Zorbax C3 column eluted at 55° C. with 0.1 M NH₄OAc as buffer A and 1.5 M NH₄Br+0.1 M NH₄OAc in 40% aqueous MeCN as buffer B using a linear gradient from 0 to 100% B for 45 min.

The purified material is acidified with AcOH to pH 4.2 and kept for 2 days. Upon neutralization with concentrated aqueous ammonium hydroxide to pH 8.0, the solution is concentrated in vacuo, extracted with ethyl acetate (2×50 mL), and dialyzed in Spectra/Pore® 2000 cellulose dialysis bags (cutoff limit 2000 Da) against water (3×1 L) and 0.1 M NaOAc (pH 7.8; 1 L). The dialyzed solution is finally desalted on a Sephadex G25 column eluting with 30% aqueous MeCN to give pure oligonucleotide-PEI 600 conjugate.

### Example 34

Conjugate of Polyethylenimine 600 with Oligonucleotide (TG)₂ctat₂c(tg)₂A₂T₂ via 3′-O-[2-[[carbamoyl]methoxy]ethyl]thiophosphate Group.

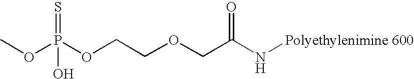

A solid support-bound oligonucleotide (TG)₂ctat₂c(tg)₂A₂T₂ assembled on 2-[2-[2-(4,4′-dimethoxytrityloxy)ethoxy]acetylthio]acetyl-derivatized CPG (4 μmol) is treated with a solution of 25% polyethylenimine and 1 M thiophenol in 30% aqueous MeCN (0.2 mL per μmol) for 1 h at room temperature. The reaction mixture was diluted with concentrated aqueous ammonium hydroxide (1 mL per μmol) and kept at 60° C. for 12 h. The solid phase was removed by filtration or centrifugation, the solution was evaporated. The residue was re-dissolved in water (1 mL per μmol) and filtered again to give crude 5′-DMT-protected oligonucleotide derivatized with PEI 600. The solution is cooled in an ice bath and neutralized with 50% aqueous AcOH. The precipitate of crude oligonucleotide conjugate complexed with excess polyethylenimine is collected by centrifugation, washed with MeCN (5×20 mL) and ether (5×20 mL), and re-dissolved in a mixture of piperidine and DMSO (9:1; 20 mL). The oligonucleotide is first purified on a Sephadex G25 column (500 mL) eluting with 30% aqueous MeCN.

The obtained product is purified on a Zorbax C3 column eluted at 55° C. with 0.1 M NH₄OAc as buffer A and 1.5 M NH₄Br+0.1 M NH₄OAc in 40% aqueous MeCN as buffer B using a linear gradient from 0 to 100% B for 45 min. The purified material is acidified with AcOH to pH 4.2 and kept for 2 days. Upon neutralization with concentrated aqueous ammonium hydroxide to pH 8.0, the solution is concentrated in vacuo, extracted with ethyl acetate (2×50 mL), and dialyzed in Spectra/Pore® 2000 cellulose dialysis bags (cutoff limit 2000 Da) against water (3×1 L) and 0.1 M NaOAc (pH 7.8; 1 L). The dialyzed solution is finally desalted on a Sephadex G25 column eluting with 30% aqueous MeCN to give pure oligonucleotide-PEI 600 conjugate.

### Example 35

Synthesis PNA Conjugated to PEI or Other Polyamines

PEI and other polyamines, such as spermine, spermidine, are attached to the N-terminus of PNA by solid phase synthesis by first modifying the N-terminus with a dicarboxylic acid anhydride. Therefore, the N-terminal protection of the PNA (Fmoc or Boc) on the resin is removed by standard procedures before a solution of 10 equiv. anhydride and 0.1 equiv. DMAP in CH2CL2/pyridine (5:1) are added and the suspension is shaken for 16 h. After washing the resin with DCM and DMF, the acid function on the resin is activated by adding a solution of HATU in DMF (10 equiv.) followed by a solution of DIEA in NMP (40 equiv.). The suspension is shaken for about 10 min, before the excess of reagents is washed out under argon atmosphere with dry DMF. Then the polyamine is added as a solution in DMF or neat in 10-100-fold excess and the suspension is shaken at rt for 16 h. After washing the resin with DMF and DCM, the PNA conjugate is cleaved from the resin according to standard procedures depending on the chemistry used (Fmoc or Boc). The isolated conjugate is purified by reversed phase HLPC according to standard protocols.

Synthesis of PNA-PEI Conjugates

TABLE 2
PNA-polyamine conjugates

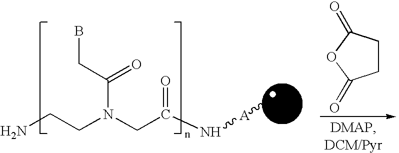

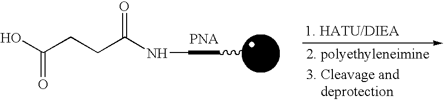

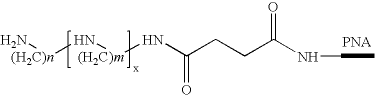

| Isis # | Sequence 5′ → 3′ | Polyamine X | Target |
|---|---|---|---|
| | X-suc-CTC AGC ACA TCT ACA- | spermine | PTEN |
| | Lys (SEQ ID NO:9) | | |
| | X-suc-CAC AGA TGA CAT TAG- | spermine | CD40 |
| | Lys (SEQ ID NO:10) | | |
| | X-suc-CTC AGC ACA TCT ACA- | spermidine | PTEN |
| | Lys (SEQ ID NO:11) | | |
| | X-suc-CAC AGA TGA CAT TAG- | spermidine | CD40 |
| | Lys (SEQ ID NO:12) | | |
| | X-suc-CTC AGC ACA TCT ACA- | PEI100-40000 | PTEN |
| | Lys (SEQ ID NO:13) | | |
| | X-suc-CAC AGA TGA CAT TAG- | PEI100-40000 | CD40 |
| | Lys (SEQ ID NO:14) | | |

suc = succinyl

We claim:

1. An oligomeric compound of formula I: 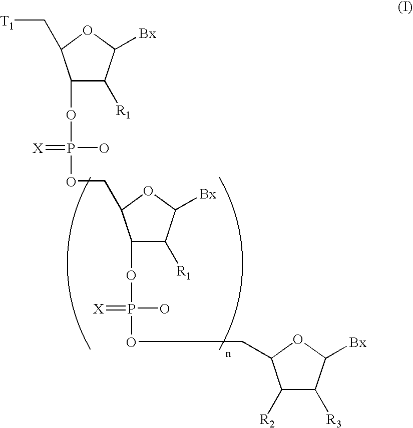
wherein:
T₁ is hydroxyl or a protected hydroxyl;
each Bx is an optionally protected heterocyclic base moiety;
each R₁ is, independently, hydrogen or a sugar substituent group;
each X is, independently, S or O;
n is from 2 to about 50;
one of R₂ and R₃ is -L-R₄, and the other of R₂ and R₃ is -L-R₄, hydrogen or a sugar substituent group;
each L is a linking group; and
R₄ is a polyethylenamino radical having a molecular weight of from about 100 daltons to about 100,000 daltons.

2. The oligomeric compound of claim 1 wherein R₄ is a polyethylenamino radical of formula II: 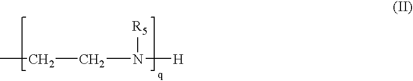
wherein:
q is from about 2 to about 1700; and
each R₅ is, independently, H or a group of formula III: 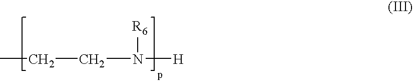
wherein:
p is from 1 to about 1000; and
each R₆ is, independently, H or a group of formula (II).

3. The oligomeric compound of claim 2 wherein each R₅ is H.

4. The oligomeric compound of claim 2 wherein at least one R₅ is a group of formula III:

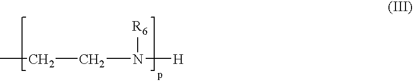

5. The oligomeric compound of claim 1 wherein each L is, independently, a linking group of formula IV: 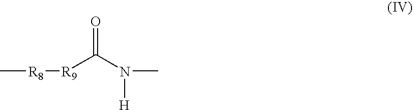
wherein:
R₈ is —O—, phosphate or phosphorothioate and is covalently attached to the R₂ or R₃ position of formula I;
R₉ is (CH₂)ₘ, (CH₂)ₘₘ—C₆-C₂₀ aryl or a polyethylene glycol —(CH₂)₂—[O—(CH₂)₂]ₘₘₘ—;
m is from 1 to about 6;
mm is from 1 to about 6; and
mmm from 1 to about 6.

6. The oligomeric compound of claim 5 wherein at least one L is a group of formula V:

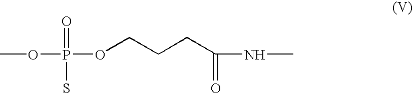

7. The oligomeric compound of claim 1 wherein R₃ is -L-R₄.

8. An oligomeric compound of formula VI: 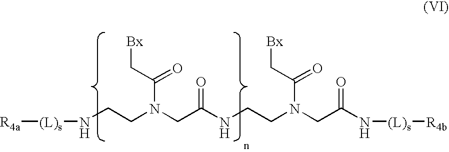
wherein:
each Bx is an optionally protected heterocyclic base moiety;
n is from 2 to about 50;
each L is a linking group;
each s is 0 or 1;
at least one of R₄ₐ and R_{4b} is a polyethylenamino radical having a molecular weight of from about 100 daltons to about 100,000 daltons, and if R₄ₐ or R_{4b} is not a polyethylenamino radical it is hydrogen, an amino protecting group, a carbonyl protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid, or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl.

9. The oligomeric compound of claim 8 wherein R₄ is a polyethylenamino radical of formula II: 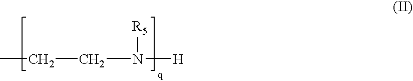
wherein:
q is from about 2 to about 1700; and
each R₅ is, independently, H or a group of formula III: 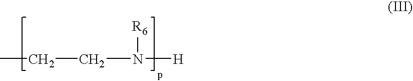
wherein:
p is from 1 to about 1000; and
each R₆ is, independently, H or a group of formula (II).

10. The oligomeric compound of claim 9 wherein each R₅ is H.

11. The oligomeric compound of claim 9 wherein at least one R₅ is a group of formula III:

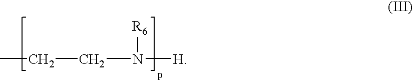

12. The oligomeric compound of claim 1 wherein each L is, independently, a linking group of formula IV: 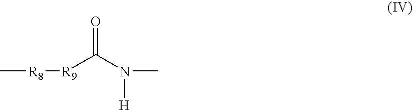
wherein:
R₈ is —O—, phosphate or phosphorothioate and is covalently attached to the R₂ or R₃ position of formula I;
R₉ is (CH₂)ₘ, (CH₂)ₘₘ—C₆-C₂₀ aryl or a polyethylene glycol —(CH₂)₂—[O—(CH₂)₂]ₘₘₘ—;
m is from 1 to about 6;
mm is from 1 to about 6; and
mmm from 1 to about 6.

13. A compound comprising an oligomeric moiety, a fusogenic moiety, and a targeting moiety.

14. The compound of claim 13 wherein the fusogenic moiety is covalently linked to the oligomeric moiety.

15. The compound of claim 14 wherein the targeting moiety is covalently linked to the oligomeric moiety.

16. The compound of claim 14 wherein the targeting moiety is covalently linked to the fusogenic moiety.

17. The compound of claim 13 wherein the fusogenic moiety is a lipophilic polyamine, polyethylenimine, polyallylamine, fusogenic peptide, oligomeric imidazole, histidine, pyridine, hydroxylamine, substituted hydroxylamine, hydrazine, substituted hydrazine, thiourea, or imine.

18. The compound of claim 17 wherein the fusogenic moiety is a polyethylenamine radical having a molecular weight of from about 100 daltons to about 100,000 daltons.

19. The compound of claim 18 wherein the polyethylenamine radical is a radical of formula (II): 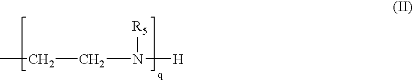
wherein:
q is from about 2 to about 1700; and
each R₅ is, independently, H or a group of formula III: 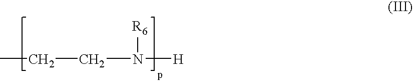
wherein:
p is from 1 to about 1000; and
each R₆ is, independently, H or a group of formula (II).

20. The compound of claim 19 wherein each R₅ is H.

21. The compound of claim 19 wherein at least one R₅ is a group of formula III:

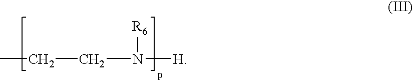

22. The compound of claim 13 wherein the targeting moiety is a ligand that binds to a cellular receptor.

23. The compound of claim 22 wherein the targeting moiety is transferrin, folate, epidermal growth factor, nerve growth factor, insulin, alpha-fetoprotein, galactose, galactosamine, lactose, mannose, a polyclonal antibody, or a moloclonal antibody.

24. The compound of claim 13 wherein the targeting moiety is Vitamin B₁₂, ibuprofen, cholesterol, or low-density lipoprotein.

25. The compound of claim 13 wherein the targeting moiety is a peptide comprising an arginine-glycine-aspartic acid sequence.

26. The compound of claim 13 wherein the oligomeric moiety is an oligonucleotide, an oligonucleotide analog, a peptide nucleic acid, or a peptide nucleic acid analog.

27. A method of enhancing the cellular uptake of an oligomeric compound comprising conjugating the oligomeric compound to a fusogenic moiety.

28. The method of claim 27 wherein the fusogenic moiety is a lipophilic polyamine, polyethylenimine, polyallylamine, fusogenic peptide, oligomeric imidazole, histidine, pyridine, hydroxylamine, substituted hydroxylamine, hydrazine, substituted hydrazine, thiourea, or imine.

29. The method of claim 28 wherein the fusogenic moiety is a polyethylenamine radical having a molecular weight of from about 100 daltons to about 100,000 daltons.

30. The method of claim 29 wherein the polyethylenamine radical is a radical of formula II: 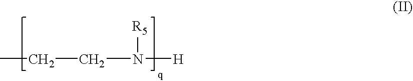
wherein:
q is from 2 to about 1700; and
each R₅ is, independently, H or a group of formula III: 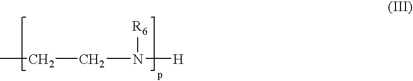
wherein:
p is from 1 to about 1000; and
each R₆ is, independently, H or a group of formula (II).

31. The method of claim 30 wherein each R₅ is H.

32. The method of claim 30 wherein at least one R₅ is a group of formula III:

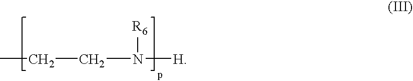

33. The method of claim 27 wherein the oligomeric compound is an oligonucleotide, an oligonucleotide analog, a peptide nucleic acid, or a peptide nucleic acid analog.

34. The method of claim 27 further comprising conjugating the oligomeric compound-fusogenic moiety conjugate to a targeting moiety.

35. The method of claim 34 wherein the targeting moiety is covalently linked to the oligomeric compound.

36. The method of claim 34 wherein the targeting moiety is covalently linked to the fusogenic moiety.

37. The method of claim 34 wherein the targeting moiety is a ligand that binds to a cellular receptor.

38. The method of claim 37 wherein the targeting moiety is transferrin, folate, epidermal growth factor, nerve growth factor, insulin, alpha-fetoprotein, galactose, galactosamine, lactose, mannose, a polyclonal antibody, or a moloclonal antibody.

39. The method of claim 34 wherein the targeting moiety is Vitamin B₁₂, ibuprofen, cholesterol, or low-density lipoprotein.

40. The method of claim 34 wherein the targeting moiety is a peptide comprising an arginine-glycine-aspartic acid sequence.
